(19) United States
(12) Patent Application Publication
(10) Pub. No.: US 2004/0072834 A1
(43) Pub. Date: Apr. 15, 2004

(54) Crystal forms of quinoxaline-2-carboxylic acid [4-carbamoyl-1-(3-fluorobenzyl)-2,7-dihydroxy-7-methyl-octyl]-amide

(75) Inventors: John C. Kath, Waterford, CT (US); Zheng J. Li, Quaker Hill, CT (US); Zhengong B. Li, East Lyme, CT (US); Eric McElroy, Groton, CT (US); Clifford N. Meltz, Niantic, CT (US); Christopher S. Poss, Baltic, CT (US)

Correspondence Address: PFIZER INC., PATENT DEPARTMENT, MS8260-1611, EASTERN POINT ROAD, GROTON, CT, 06340, US

(73) Assignee: Pfizer Inc.

(21) Appl. No.: 10/637,475

(22) Filed: Aug. 8, 2003

Related U.S. Application Data
(60) Provisional application No. 60/403,216, filed on Aug. 12, 2002.

(51) Int. Cl. A61K 31/498; C07D 241/36

(52) U.S. Cl. 514/249; 544/355

(57) ABSTRACT

This invention relates to crystal forms of quinoxaline-2-carboxylic acid [4-carbamoyl-1-(3-fluorobenzyl)-2,7-dihydroxy-7-methyl-octyl]-amide, useful in treating or preventing a disorder or condition by antagonizing the CCR1 receptor, and to their methods of preparation and use.

### RELATED APPLICATION

The present application claims priority to U.S. patent application Ser. No. 60/403,216, filed Aug. 12, 2002, which is incorporated herein in its entirety for all purposes.

### FIELD OF THE INVENTION

This invention relates to crystal forms of quinoxaline-2-carboxylic acid [4carbamoyl-1-(3-fluorobenzyl)-2,7-dihydroxy-7-methyl-octyl]-amide and their methods of preparation and use.

### BACKGROUND OF THE INVENTION

Quinoxaline-2-carboxylic acid [4-carbamoyl-1-(3-fluorobenzyl)-2,7-dihydroxy-7-methyl-octyl]-amide has the chemical formula C₂₆H₃₁FN₄O₄ and the following structural formula (Ia-3):

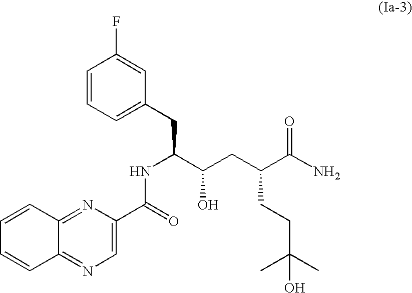

Its synthesis is described in co-pending U.S. patent application Ser. No. 09/380,269, filed Feb. 5, 1998 and U.S. patent application Ser. No. 09/403,218, filed Jan. 18, 1999, commonly assigned to the assignee of the present invention and both of which are incorporated herein by reference in their entireties for all purposes.

Quinoxaline-2-carboxylic acid [4-carbamoyl-1-(3-fluorobenzyl)-2,7-dihydroxy-7-methyl-octyl]-amide is useful in the treatment or prevention of autoimmune diseases (such as rheumatoid arthritis, type I diabetes (recent onset), inflammatory bowel disease, optic neuritis, psoriasis, multiple sclerosis, polymyalgia rheumatica, uveitis, and vasculitis), acute and chronic inflammatory conditions (such as osteoarthritis, adult Respiratory Distress Syndrome, Respiratory Distress Syndrome of infancy, ischemia reperfusion injury and glmerulonephritis), allergic conditions (such as asthma and atopic dermatitis), infection associated with inflammation (such as viral inflammation (including influenza and hepatitis) and Guillian-Barre), transplantation tissue rejection (chronic and acute), organ rejection (chronic and acute), atherosclerosis, restenosis, HIV infectivity (co-receptor usage), and granulomatous diseases (including sarcoidosis, leprosy and tuberculosis).

### SUMMARY OF THE INVENTION

As embodied and broadly described herein, this invention, in one aspect, relates to crystal forms of quinoxaline-2-carboxylic acid [4-carbamoyl-1-(3-fluoro-benzyl)-2,7-dihydroxy-7-methyl-octyl]-amide form A having a powder X-ray diffraction pattern comprising peaks expressed in degrees two-theta at approximately 5.1, 8.8, 10.1, 13.3, 15.1, 17.5, 18.2, 19.5, 20.2, 20.8, 22.0, 22.6, 23.2, 24.2, 25.3, 26.3, 26.8, 28.2, 33.3, and 38.6.

In one preferred embodiment of this aspect of the invention, the crystal forms of quinoxaline-2-carboxylic acid [4-carbamoyl-1-(3-fluoro-benzyl)-2,7-dihydroxy-7methyl-octyl]-amide have powder X-ray diffraction pattern comprising high intensity peaks expressed in degrees two-theta at approximately 10.1, 13.3, 17.5, 18.2, and 22.0.

A second aspect of the present invention relates to crystal forms of quinoxaline-2-carboxylic acid [4-carbamoyl-1-(3-fluoro-benzyl)-2,7-dihydroxy-7methyl-octyl]-amide having a solid state nuclear magnetic resonance spectra pattern comprising chemical shifts expressed in parts per million at approximately 39.0, 38.4, 32.6, 30.4, 28.5, and 26.4.

In a preferred embodiment of the invention, the crystal forms of quinoxaline-2-carboxylic acid [4-carbamoyl-1-(3-fluoro-benzyl)-2,7-dihydroxy-7-methyl-octyl]-amide have a differential scanning calorimetry thermogram comprising an endothermic event with an onset temperature approximately 139° C. using a heating rate of about 5° C. per minute from about 30° C. to about 300° C.

A third aspect of the present invention relates to crystal forms of quinoxaline-2-carboxylic acid [4-carbamoyl-1-(3-fluoro-benzyl)-2,7-dihydroxy-7-methyl-octyl]-amide form B having a powder X-ray diffraction pattern comprising peaks expressed in degrees two-theta at approximately 6.0, 7.4, 11.0, 13.8, 14.2, 14.8, 15.3, 15.7, 16.1, 16.6, 17.8, 18.6, 19.3, 20.9, 21.1, 21.6, 22.1, 23.1, 25.0, 26.1, 27.0, 27.3, 28.1, 28.7, 29.7, 31.2, and 32.4.

In a fourth aspect, the present invention relates to crystal forms of quinoxaline-2-carboxylic acid [4-carbamoyl-1-(3-fluoro-benzyl)-2,7-dihydroxy-7methyl-octyl]-amide having a solid state nuclear magnetic resonance spectra pattern comprising chemical shifts expressed in parts per million at approximately 40.9, 38.3, 34.8, 31.4, and 26.4.

In a preferred embodiment, the crystal forms of quinoxaline-2-carboxylic acid [4-carbamoyl-1-(3-fluoro-benzyl)-2,7-dihydroxy-7-methyl-octyl]-amide have a differential scanning calorimetry thermogram comprising an endothermic event with an onset temperature at approximately 160° C. using a heating rate of about 5° C. per minute from about 30° C. to about 300° C.

In a fifth aspect, the present invention relates to forms of quinoxaline-2-carboxylic acid [4-carbamoyl-1-(3-fluoro-benzyl)-2,7-dihydroxy-7-methyl-octyl]-amide form C having a powder X-ray diffraction pattern comprising peaks expressed in degrees two-theta at approximately 4.6, 7.4, 8.4, 10.8, 11.9, 12.6, 13.4, 14.1, 14.8, 15.6, 16.4, 17.4, 17.8, 18.1, 18.7, 19.0, 19.7, 20.6, 21.1, 21.7, 22.1, 22.6, 23.1, 24.1, 24.5, 25.0, 25.6, 26.2, 27.3, 27.7, 28.3, 29.0, 30.3, 30.6, 31.0, 32.1, 32.6, 33.3, 34.1, 34.4, 35.4, 35.7, 37.2, 38.4, and 39.3.

One preferred embodiment includes crystal forms of quinoxaline-2-carboxylic acid [4-carbamoyl-1-(3-fluoro-benzyl)-2,7-dihydroxy-7-methyl-octyl]-amide having a differential scanning calorimetry thermogram comprising an endothermic event with an onset temperature of about 154° C. using a heating rate of about 5° C. per minute from about 30° C. to about 300° C.

A sixth aspect of the present invention relates to crystal forms of quinoxaline-2-carboxylic acid [4-carbamoyl-1-(3-fluoro-benzyl)-2,7-dihydroxy-7-methyl-octyl]-amide form D having a powder X-ray diffraction pattern comprising peaks expressed in degrees two-theta at 6.0, 7.3, 8.1, 8.6, 10.0, 10.3, 10.7, 12.1, 12.5, 13.2, 13.5, 15.1, 15.9, 16.8, 17.4, 17.8, 18.2, 18.8, 19.4, 20.0, 20.8, 21.1, 21.8, 22.0, 22.9, 23.7 24.4, 25.0, 25.4, 25.7, 26.3, 27.0, 27.5, 29.7, 30.3, 32.1, 35.4, and 36.9.

A preferred embodiment includes crystal forms of quinoxaline-2-carboxylic acid [4-carbamoyl-1-(3-fluoro-benzyl)-2,7-dihydroxy-7-methyl-octyl]-amide having a differential scanning calorimetry thermogram comprising an endothermic event with an onset temperature of about 156° C. using a heating rate of about 5° C. per minute from about 30° C. to about 300° C.

In a seventh aspect, the present invention relates to crystal forms of quinoxaline-2-carboxylic acid [4-carbamoyl-1-(3-fluoro-benzyl)-2,7-dihydroxy-7methyl-octyl]-amide form E having a powder X-ray diffraction pattern comprising peaks expressed in degrees two-theta at approximately 5.9, 7.6, 9.2, 12.0, 13.9, 14.3, 15.2, 16.0, 16.6, 17.3, 17.7, 18.0, 18.5, 19.4, 20.1, 20.6, 21.2, 21.9, 22.3, 22.8, 23.4, 24.3, 24.9, 25.4, 26.0, 26.5, 28.0, 28.7, 29.2, 29.8, 30.9, 32.3, 33.6, 33.9, 35.6, 37.3, and 37.6.

One preferred embodiment includes crystal forms of quinoxaline-2-carboxylic acid [4-carbamoyl-1-(3-fluoro-benzyl)-2,7-dihydroxy-7-methyl-octyl]-amide having powder X-ray diffraction patterns comprising high intensity peaks expressed in degrees two-theta at approximately 15.2, 16.6, 18.5, 20.6, and 21.2.

In an eighth aspect, the present invention relates to crystal forms of quinoxaline-2-carboxylic acid [4-carbamoyl-1-(3-fluoro-benzyl)-2,7-dihydroxy-7methyl-octyl]-amide having a solid state nuclear magnetic resonance spectra pattern comprising chemical shifts expressed in parts per million at approximately 40.8, 37.3, 35.5, 30.4, 27.6, and 26.0.

Another preferred embodiment of the invention includes crystal forms of quinoxaline-2-carboxylic acid [4-carbamoyl-1-(3-fluoro-benzyl)-2,7-dihydroxy-7methyl-octyl]-amide having a differential scanning calorimetry thermogram comprising an endothermic event with an onset temperature of about 163° C. using a heating rate of about 5° C. per minute from about 30° C. to about 300° C.

A ninth aspect of the present invention relates to crystal forms of quinoxaline-2-carboxylic acid [4-carbamoyl-1-(3-fluoro-benzyl)-2,7-dihydroxy-7-methyl-octyl]-amide Form F having a powder X-ray diffraction pattern comprising peaks expressed in degrees two-theta at approximately 5.4, 7.8, 10.8, 14.7, 15.6, 15.9, 16.6, 17.4, 18.1, 18.7, 20.1, 20.6, 21.8, 22.3, 24.2, 25.4, 25.8, 26.6, 29.8, and 31.4.

In a preferred embodiment of the present invention, the crystal form of quinoxaline-2-carboxylic acid [4-carbamoyl-1-(3-fluoro-benzyl)-2,7-dihydroxy-7methyl-octyl]-amide have a differential scanning calorimetry thermogram comprising an endothermic event with an onset temperature of about 188° C. using a heating rate of about 5° C. per minute from about 30° C. to about 300° C.

In a tenth aspect, the present invention relates to crystal forms of quinoxaline2-carboxylic acid [4-carbamoyl-1-(3-fluoro-benzyl)-2,7-dihydroxy-7-methyl-octyl]-amide comprising form A, form B, form C, form D, form E or form F.

In still another aspect, the present invention relates to crystal forms of quinoxaline-2-carboxylic acid [4-carbamoyl-1-(3-fluoro-benzyl)-2,7-dihydroxy-7-methyl-octyl]-amide, wherein the crystal has an empirical formula of C₂₆H₃₁N₄O₄F; a formula weight of about 482.55; a melt temperature of about 298(2) K; wavelength of about 1.54178 Å; orthorhombic crystal system; a space group P2(1)2(1)2(1); unit cell dimensions of a about 6.7678(2) Å α=90°, b about 12.6136(3) Å β=90°, and c about 29.4200(7) Å γ=90°; volume of about 2511.48(11) Å³ and Z of 4.

In preferred embodiments, the present invention includes pharmaceutical compositions for treating or preventing a disorder or condition that can be treated or prevented by antagonizing the CCR1 receptor in a subject, comprising an amount of a compound of any of the aforementioned aspects, effective in such disorders or conditions, or a pharmaceutically acceptable salt thereof, and a pharmaceutically acceptable carrier.

In further preferred embodiments, the present invention includes pharmaceutical compositions for treating or preventing a disorder or condition selected from autoimmune diseases, acute and chronic inflammatory conditions, allergic conditions, infection associated with inflammation, viral, transplantation tissue rejection, atherosclerosis, restenosis, HIV infectivity, and granulomatous in a subject, comprising an amount of a compound of any of the aforementioned aspects, effective in such disorders or conditions, or a pharmaceutically acceptable salt thereof, and a pharmaceutically acceptable carrier.

Moreover, the present invention relates to methods for treating or preventing a disorder or condition that can be treated or prevented by antagonizing the CCR1 receptor in a subject, comprising administering to said subject an effective amount of a compound of any of the aforementioned aspects of the present invention.

In a further aspect, the present invention relates to methods for treating or preventing a disorder or condition selected from autoimmune diseases, acute and chronic inflammatory conditions, allergic conditions, infection associated with inflammation, viral, transplantation tissue rejection, atherosclerosis, restenosis, HIV infectivity, and granulomatous in a subject, comprising administering to said subject an effective amount of a compound of any of the aforementioned aspects of the present invention.

In yet another aspect, the present invention relates to methods of preparing crystalline quinoxaline-2-carboxylic acid [4-carbamoyl-1-(3-fluorobenzyl)-2,7dihydroxy-7-methyl-octyl]-amide comprising: a) mixing quinoxaline-2-carboxylic acid [4-carbamoyl-1-(3-fluorobenzyl)-2,7-dihydroxy-7-methyl-octyl]-amide free base in a solvent mixture of methanol and methylene chloride to create mixture 1; b) distilling mixture 1 to substantially remove methanol to form mixture 2; and c) crystallizing mixture 2 in a solvent system comprising ethyl acetate. Preferably, the solvent system further comprises methanol, and the step (c) is performed by creating a slurry of mixture 2 in the solvent system and substantially removing the methanol by distillation.

It is to be understood that both the foregoing general description and the following detailed description are exemplary and explanatory only and are not restrictive of the invention, as claimed.

### BRIEF DESCRIPTION OF THE DRAWINGS

FIG. 1 is a representative powder X-ray diffraction pattern for quinoxaline-2-carboxylic acid [4-carbamoyl-1-(3-fluorobenzyl)-2,7-dihydroxy-7-methyl-octyl]-amide, form A, (Vertical Axis: Intensity (counts); Horizontal Axis: Two Theta (Degrees)).

FIG. 2 is a representative differential scanning calorimetry thermogram of quinoxaline-2-carboxylic acid [4-carbamoyl-1-(3-fluorobenzyl)-2,7-dihydroxy-7-methyl-octyl]-amide, form A, (Scan Rate: 5° C. per minute; Vertical Axis: Heat Flow (mW); Horizontal Axis: Temperature (° C.)).

FIG. 3 is a representative powder X-ray diffraction pattern for quinoxaline-2-carboxylic acid [4-carbamoyl-1-(3-fluorobenzyl)-2,7-dihydroxy-7-methyl-octyl]-amide, form B, (Vertical Axis: Intensity (counts); Horizontal Axis: Two Theta (Degrees)).

FIG. 4 is a representative differential scanning calorimetry thermogram of quinoxaline-2-carboxylic acid [4-carbamoyl-1-(3-fluorobenzyl)-2,7-dihydroxy-7methyl-octyl]-amide, form B, (Scan Rate: 5° C. per minute; Vertical Axis: Heat Flow (mW); Horizontal Axis: Temperature (° C.)).

FIG. 5 is a representative powder X-ray diffraction pattern for quinoxaline-2-carboxylic acid [4-carbamoyl-1-(3-fluorobenzyl)-2,7-dihydroxy-7-methyl-octyl]-amide, form C, (Vertical Axis: Intensity (counts); Horizontal Axis: Two Theta (Degrees)).

FIG. 6 is a representative differential scanning calorimetry thermogram of quinoxaline-2-carboxylic acid [4-carbamoyl-1-(3-fluorobenzyl)-2,7-dihydroxy-7methyl-octyl]-amide, form C, (Scan Rate: 5° C. per minute; Vertical Axis: Heat Flow (mW); Horizontal Axis: Temperature (° C.)).

FIG. 7 is a representative powder X-ray diffraction pattern for quinoxaline-2-carboxylic acid [4-carbamoyl-1-(3-fluorobenzyl)-2,7-dihydroxy-7-methyl-octyl]-amide, form D, (Vertical Axis: Intensity (counts); Horizontal Axis: Two Theta (Degrees)).

FIG. 8 is a representative differential scanning calorimetry thermogram of quinoxaline-2-carboxylic acid [4-carbamoyl-1-(3-fluorobenzyl)-2,7-dihydroxy-7-methyl-octyl]-amide, form D, (Scan Rate: 5° C. per minute; Vertical Axis: Heat Flow (mW); Horizontal Axis: Temperature (° C.)).

FIG. 9 is a representative powder X-ray diffraction pattern for quinoxaline-2-carboxylic acid [4-carbamoyl-1-(3-fluorobenzyl)-2,7-dihydroxy-7-methyl-octyl]-amide, form E, (Vertical Axis: Intensity (counts); Horizontal Axis: Two Theta (Degrees)).

FIG. 10 is a representative differential scanning calorimetry thermogram of quinoxaline-2-carboxylic acid [4-carbamoyl-1-(3-fluorobenzyl)-2,7-dihydroxy-7-methyl-octyl]-amide, form E, (Scan Rate: 5° C. per minute; Vertical Axis: Heat Flow (mW); Horizontal Axis: Temperature (° C.)).

FIG. 11 is a representative powder X-ray diffraction pattern for quinoxaline-2-carboxylic acid [4-carbamoyl-1-(3-fluorobenzyl)-2,7-dihydroxy-7-methyl-octyl]-amide, form F, (Vertical Axis: Intensity (counts); Horizontal Axis: Two Theta (Degrees)).

FIG. 12 is a representative differential scanning calorimetry thermogram of quinoxaline-2-carboxylic acid [4-carbamoyl-1-(3-fluorobenzyl)-2,7-dihydroxy-7methyl-octyl]-amide, form F, (Scan Rate: 5° C. per minute; Vertical Axis: Heat Flow (mW); Horizontal Axis: Temperature (° C.)).

FIG. 13 depicts the calculated and representative powder X-ray diffraction patterns of quinoxaline-2-carboxylic acid [4-carbamoyl-1-(3-fluorobenzyl)-2,7dihydroxy-7-methyl-octyl]-amide, form E, (Vertical Axis: Intensity (counts); Horizontal Axis: Two Theta (Degrees)).

FIG. 14 is a representative ¹³C solid state nuclear magnetic resonance spectrum for quinoxaline-2-carboxylic acid [4-carbamoyl-1-(3-fluorobenzyl)-2,7dihydroxy-7-methyl-octyl]-amide, form A, (Vertical Axis: Intensity (counts); Horizontal Axis: Chemical shift (δ-scale), in ppm).

FIG. 15 is a representative ¹³C solid state nuclear magnetic resonance spectrum for quinoxaline-2-carboxylic acid [4-carbamoyl-1-(3-fluorobenzyl)-2,7-dihydroxy-7-methyl-octyl]-amide, form B, (Vertical Axis: Intensity (counts); Horizontal Axis: Chemical shift (δ-scale), in ppm).

FIG. 16 is a representative ¹³C solid state nuclear magnetic resonance spectrum for quinoxaline-2-carboxylic acid [4-carbamoyl-1-(3-fluorobenzyl)-2,7dihydroxy-7-methyl-octyl]-amide, form E, (Vertical Axis: Intensity (counts); Horizontal Axis: Chemical shift (δ-scale), in ppm).

FIG. 17 depicts the absolute configuration of Form E as derived from single crystal X-ray. (Atomic coordinates based on Tables 1-B, 1-C and 1-D.

### DETAILED DESCRIPTION OF THE INVENTION

The present invention may be understood more readily by reference to the following detailed description of exemplary embodiments of the invention and the examples included therein.

Before the present crystal forms and methods are disclosed and described, it is to be understood that this invention is not limited to specific synthetic methods of making that may of course vary. It is also to be understood that the terminology used herein is for the purpose of describing particular embodiments only and is not intended to be limiting.

In this specification and in the claims that follow, reference will be made to a number of terms that shall be defined to have the following meanings:

By “pharmaceutically acceptable” is meant a material that is not biologically or otherwise undesirable, i.e., the material may be administered to an individual along with the selected compound without causing any undesirable biological effects or interacting in a deleterious manner with any of the other components of the pharmaceutical composition in which it is contained.

“Protected amine” and “protected amino” refers to an amine group with one of the hydrogen atoms replaced with a protecting group (P). Any suitable protecting group may be used for amine protection. Suitable protecting groups include, but are not limited to, carbobenzyloxy, t-butoxy carbonyl or 9-fluorenyl-methylenoxy carbonyl.

The term “subject” is meant an individual. Preferably, the subject is a mammal such as a primate, and more preferably, a human. Thus, the “subject” can include domesticated animals (e.g., cats, dogs, etc.), livestock (e.g., cattle, horses, pigs, sheep, goats, etc.), and laboratory animals (e.g., mouse, rabbit, rat, guinea pig, etc.).

In general, “effective amount” or “effective dose” means the amount needed to achieve the desired result or results (treating or preventing the condition). One of ordinary skill in the art will recognize that the potency and, therefore, an “effective amount” can vary for the various compounds used in the invention. One skilled in the art can readily assess the potency of the compounds.

Unless otherwise noted, numerical values described and claimed herein are approximate. Variation within the values may be attributed to equipment calibration, equipment errors, purity of the materials, crystal size, and sample size, among other factors. Additionally, variation may be possible, while still obtaining the same result. For example, X-ray diffraction values are generally accurate to within ±0.2 2-theta degrees, preferably to within ±0.2 2-theta degrees. Similarly, DSC results are typically accurate to within about 2° C., preferably to within 1.5° C. Also, ¹³C ss-NMR results are generally accurate to within about ±0.2 ppm.

The crystalline state of a compound can be described by several crystallographic parameters including single crystal structure and powder crystal X-ray diffraction pattern. Such crystalline description is advantageous because a compound may have more than one type of crystal form. It has been discovered that there are at least six crystal forms (Forms A, B, C, D, E, and F) of quinoxaline-2-carboxylic acid [4-carbamoyl-1-(3-fluoro-benzyl)-2,7-dihydroxy-7-methyl-octyl]-amide.

To describe and distinguish the crystal forms, several crystal parameters are identified in the present invention: Form E has been examined by single crystal X-ray analysis; Forms A-F have been examined by powder X-ray diffraction and differential scanning calorimetry (DSC); and Forms A, B and E have been examined by solid state Nuclear Magnetic Resonance (NMR). A discussion of the theory of X-ray power diffraction patterns can be found in Stout & Jensen, X-Ray Structure Determination; A Practical Guide, MacMillan Co., New York, N.Y. (1968), which is incorporated by reference in its entirety for all purposes.

Form E is the thermodynamically most stable crystal form at room temperature and is one preferred crystal form for tablet development. A representative crystal was surveyed and a 1 Å data set (maximum sin Θ/λ=0.5) was collected on a Bruker 2k CCD/R diffractometer. Atomic scattering factors were taken from the International Tables for X-Ray Crystalloagraphy. (International Tables for X-Ray Crystallography, Vol. IV, pp. 55,99,149 Birmingham: Kynoch Press, 1974.) All crystallographic calculations were facilitated by the SHELXTL system. (SHELXTL, Version 5.1, Bruker AXS,1997.) All diffractometer data were collected at room temperature. Pertinent crystal, data collection, and refinement are summarized in Table 1-A.

A trial structure was obtained by direct methods. This trial structure refined routinely. Hydrogen positions were calculated wherever possible. The methyl hydrogens and the hydrogens on nitrogen and oxygen were located by difference Fourier techniques. The hydrogen parameters were added to the structure factor calculations but were not refined. The shifts calculated in the final cycles of least squares refinement were all less than 0.1 of the corresponding standard deviations. The final R-index was 3.36%. A final difference Fourier revealed no missing or misplaced electron density.

The refined structure was plotted using the SHELXTL plotting package; however, the absolute configuration was not determined in this analysis because no suitable “heavy atom” was present in the structure. Coordinates, distances and angles are available in Tables 1B through 1D.

TABLE 1 A
Single Crystal X-ray Crystallographic Analysis of Form E

| | | |
|---|---|---|
| Identification code | F804 | |
| Empirical formula | C₂₆H₃₁N₄O₄F | |
| Formula weight | 482.55 | |
| Temperature | 298(2) K | |
| Wavelength | 1.54178 Å | |
| Crystal system | Orthorhombic | |
| Space group | P2(1)2(1)2(1) | |
| Unit cell dimensions | a = 6.7678(2) Å | □ = 90°. |
| | b = 12.6136(3) Å | □ = 90°. |
| | c = 29.4200(7) Å | □ = 90°. |
| Volume | 2511.48(11)Å³ | |
| Z (number of chemical formula units | 4 | |
| per unit cell) | | |
| Density (calculated) | 1.276 Mg/m³ | |
| Absorption coefficient | 0.759 mm⁻¹ | |
| F(000) | 1024 | |
| Crystal size | 0.03 × 0.06 × 0.15 mm³ | |
| Reflections collected | 5416 | |
| Independent reflections | 2024 [R(int) = 0.0519] | |
| Absorption correction | None | |
| Refinement method | Full-matrix least-squares on F² | |
| Data / restraints / parameters | 2024 / 0 / 329 | |
| Goodness-of-fit on F² | 0.782 | |
| Final R indices [I > 2sigma(I)] | R1 = 0.0336, wR2 = 0.0789 | |
| Absolute structure parameter | 0.4(3) | |
| Extinction coefficient | 0.0026(3) | |
| Largest diff. peak and hole | 0.096 and −0.101 e.Å⁻³ | |

TABLE 1 B
Atomic coordinates (×10⁴) and equivalent isotropic displacement
parameters (Å² × 10³) for form E. U(eq)
is defined as one third of the trace of the
orthogonalized Uᵢⱼ tensor.

| | x | y | z | U(eq) |
|---|---|---|---|---|
| N(1) | 1299(5) | 2603(3) | 828(1) | 48(1) |
| C(2) | 3009(7) | 2115(3) | 868(1) | 45(1) |
| C(3) | 4671(6) | 2424(4) | 612(2) | 60(1) |
| N(4) | 4643(5) | 3211(3) | 319(1) | 64(1) |
| C(5) | 2878(7) | 3731(3) | 269(1) | 52(1) |
| C(6) | 1209(7) | 3430(3) | 530(1) | 48(1) |
| C(7) | −555(7) | 3985(3) | 473(1) | 65(1) |
| C(8) | −650(7) | 4792(4) | 172(2) | 77(1) |
| C(9) | 971(9) | 5091(3) | −87(2) | 79(1) |
| C(10) | 2710(7) | 4578(4) | −36(1) | 67(1) |
| C(11) | 3206(7) | 1213(3) | 1196(1) | 49(1) |
| O(12) | 4771(4) | 748(2) | 1249(1) | 73(1) |
| N(13) | 1540(5) | 969(2) | 1416(1) | 52(1) |
| C(14) | 1401(5) | 148(3) | 1764(1) | 48(1) |
| C(15) | −621(6) | −385(3) | 1740(1) | 50(1) |
| O(16) | −2059(4) | 387(2) | 1858(1) | 73(1) |
| C(17) | −1070(5) | −883(3) | 1283(1) | 51(1) |
| C(18) | 417(5) | −1703(3) | 1113(1) | 44(1) |
| C(19) | −146(8) | −2034(3) | 634(1) | 51(1) |
| N(20) | 1345(6) | −2001(3) | 333(1) | 69(1) |
| O(21) | −1830(5) | −2301(2) | 532(1) | 65(1) |
| C(22) | 520(5) | −2695(3) | 1413(1) | 56(1) |
| C(23) | 1906(5) | −3584(3) | 1250(1) | 58(1) |
| C(24) | 4118(6) | −3387(3) | 1284(1) | 56(1) |
| O(25) | 4556(4) | −2538(2) | 964(1) | 73(1) |
| C(26) | 4746(5) | −3050(3) | 1754(1) | 83(1) |
| C(27) | 5211(6) | −4373(3) | 1133(1) | 84(1) |
| C(28) | 1868(6) | 620(3) | 2236(1) | 59(1) |
| C(29) | 2164(7) | −221(3) | 2587(1) | 48(1) |
| C(30) | 3962(7) | −717(4) | 2632(1) | 60(1) |
| C(31) | 4163(8) | −1510(4) | 2943(2) | 74(1) |
| C(32) | 2671(11) | −1843(4) | 3218(2) | 88(2) |
| C(33) | 878(9) | −1349(5) | 3173(2) | 89(2) |
| C(34) | 618(7) | −556(4) | 2864(2) | 67(1) |
| F(35) | 5963(5) | −1976(2) | 2992(1) | 128(1) |

TABLE 1 C
Bond lengths [Å] and angles [°] for form E.

| | |
|---|---|
| N(1)—C(2) | 1.316(4) |
| N(1)—C(6) | 1.364(4) |
| C(2)—C(3) | 1.409(5) |
| C(2)—C(11) | 1.497(5) |
| C(3)—N(4) | 1.316(5) |
| N(4)—C(5) | 1.370(4) |
| C(5)—C(6) | 1.418(5) |
| C(5)—C(10) | 1.400(5) |
| C(6)—C(7) | 1.394(5) |
| C(7)—C(8) | 1.350(5) |
| C(8)—C(9) | 1.389(6) |
| C(9)—C(10) | 1.352(5) |
| C(11)—O(12) | 1.221(4) |
| C(11)—N(13) | 1.336(4) |
| N(13)—C(14) | 1.459(4) |
| C(14)—C(15) | 1.526(5) |
| C(14)—C(28) | 1.545(5) |
| C(15)—O(16) | 1.419(4) |
| C(15)—C(17) | 1.516(4) |
| C(17)—C(18) | 1.528(4) |
| C(18)—C(19) | 1.518(5) |
| C(18)—C(22) | 1.533(4) |
| C(19)—O(21) | 1.226(4) |
| C(19)—N(20) | 1.342(5) |
| C(22)—C(23) | 1.538(5) |
| C(23)—C(24) | 1.521(5) |
| C(24)—O(25) | 1.456(5) |
| C(24)—C(26) | 1.507(5) |
| C(24)—C(27) | 1.514(5) |
| C(28)—C(29) | 1.494(5) |
| C(29)—C(34) | 1.392(5) |
| C(29)—C(30) | 1.374(5) |
| C(30)—C(31) | 1.364(6) |
| C(31)—F(35) | 1.360(5) |
| C(31)—C(32) | 1.359(6) |
| C(32)—C(33) | 1.370(6) |
| C(33)—C(34) | 1.363(6) |
| C(2)—N(1)—C(6) | 117.1(3) |
| N(1)—C(2)—C(3) | 121.6(3) |
| N(1)—C(2)—C(11) | 119.5(4) |
| C(3)—C(2)—C(11) | 118.9(4) |
| N(4)—C(3)—C(2) | 123.3(4) |
| C(3)—N(4)—C(5) | 116.3(3) |
| N(4)—C(5)—C(6) | 120.6(4) |
| N(4)—C(5)—C(10) | 120.3(5) |
| C(6)—C(5)—C(10) | 119.1(4) |
| N(1)—C(6)—C(5) | 121.1(4) |
| N(1)—C(6)—C(7) | 120.0(4) |
| C(5)—C(6)—C(7) | 118.8(4) |
| C(8)—C(7)—C(6) | 119.9(4) |
| C(7)—C(8)—C(9) | 121.8(4) |
| C(10)—C(9)—C(8) | 119.8(4) |
| C(9)—C(10)—C(5) | 120.5(4) |
| O(12)—C(11)—N(13) | 124.0(4) |
| O(12)—C(11)—C(2) | 121.7(4) |
| N(13)—C(11)—C(2) | 114.3(4) |
| C(11)—N(13)—C(14) | 123.9(3) |
| N(13)—C(14)—C(15) | 109.8(3) |
| N(13)—C(14)—C(28) | 110.1(3) |
| C(15)—C(14)—C(28) | 113.2(3) |
| O(16)—C(15)—C(14) | 107.5(3) |
| O(16)—C(15)—C(17) | 111.3(3) |
| C(14)—C(15)—C(17) | 113.7(3) |
| C(15)—C(17)—C(18) | 116.1(3) |
| C(19)—C(18)—C(17) | 109.0(3) |
| C(19)—C(18)—C(22) | 108.7(3) |
| C(17)—C(18)—C(22) | 113.2(3) |
| O(21)—C(19)—N(20) | 123.1(4) |
| O(21)—C(19)—C(18) | 122.4(4) |
| N(20)—C(19)—C(18) | 114.5(4) |
| C(18)—C(22)—C(23) | 116.4(3) |
| C(24)—C(23)—C(22) | 117.4(3) |
| O(25)—C(24)—C(26) | 109.2(3) |
| O(25)—C(24)—C(27) | 108.3(3) |
| C(26)—C(24)—C(27) | 111.3(3) |
| O(25)—C(24)—C(23) | 106.2(3) |
| C(26)—C(24)—C(23) | 112.6(3) |
| C(27)—C(24)—C(23) | 109.1(3) |
| C(29)—C(28)—C(14) | 112.1(3) |
| C(34)—C(29)—C(30) | 118.1(4) |
| C(34)—C(29)—C(28) | 121.3(4) |
| C(30)—C(29)—C(28) | 120.5(4) |
| C(31)—C(30)—C(29) | 119.1(4) |
| F(35)—C(31)—C(30) | 118.5(6) |
| F(35)—C(31)—C(32) | 118.0(6) |
| C(30)—C(31)—C(32) | 123.4(5) |
| C(33)—C(32)—C(31) | 117.5(5) |
| C(32)—C(33)—C(34) | 120.8(5) |
| C(33)—C(34)—C(29) | 121.1(4) |

Symmetry transformations used to generate equivalent atoms:

TABLE 1 D
Hydrogen coordinates (×10⁴) and isotropic displacement parameters
(Å² × 10 ³) for form E.

| | x | y | z | U(eq) |
|---|---|---|---|---|
| H(3A) | 5846 | 2054 | −653 | 80 |
| H(7A) | −1665 | 3800 | 641 | 80 |
| H(8A) | −1834 | 5158 | 138 | 80 |
| H(9A) | 860 | 5642 | −296 | 80 |
| H(10A) | 3804 | 4790 | −205 | 80 |
| H(13A) | 488 | 1317 | 1349 | 80 |
| H(14A) | 2404 | −391 | 1697 | 80 |
| H(15A) | −657 | −946 | 1971 | 80 |
| H(16A) | −2960(60) | 420(40) | 1699(14) | 80 |
| H(17A) | −1163 | −322 | 1059 | 80 |
| H(17B) | −2357 | −1220 | 1301 | 80 |
| H(18A) | 1729 | −1374 | 1104 | 80 |
| H(20A) | 1030(60) | −2280(30) | 69(13) | 80 |
| H(20B) | 2720(60) | −1900(30) | 440(12) | 80 |
| H(22A) | −802 | −2986 | 1439 | 80 |
| H(22B) | 932 | −2481 | 1715 | 80 |
| H(23A) | 1604 | −4218 | 1424 | 80 |
| H(23B) | 1597 | −3735 | 935 | 80 |
| H(25A) | 5700(60) | −2540(30) | 873(12) | 80 |
| H(26A) | 6142 | −2914 | 1756 | 80 |
| H(26B) | 4446 | −3604 | 1967 | 80 |
| H(26C) | 4049 | −2416 | 1838 | 80 |
| H(27A) | 6609 | −4247 | 1147 | 80 |
| H(27B) | 4844 | −4543 | 826 | 80 |
| H(27C) | 4875 | −4953 | 1329 | 80 |
| H(28A) | 3053 | 1049 | 2216 | 80 |
| H(28B) | 790 | 1078 | 2329 | 80 |
| H(30A) | 5027 | −514 | 2452 | 80 |
| H(32A) | 2859 | −2385 | 3428 | 80 |
| H(33A) | −174 | −1558 | 3355 | 80 |
| H(34A) | −612 | −234 | 2838 | 80 |

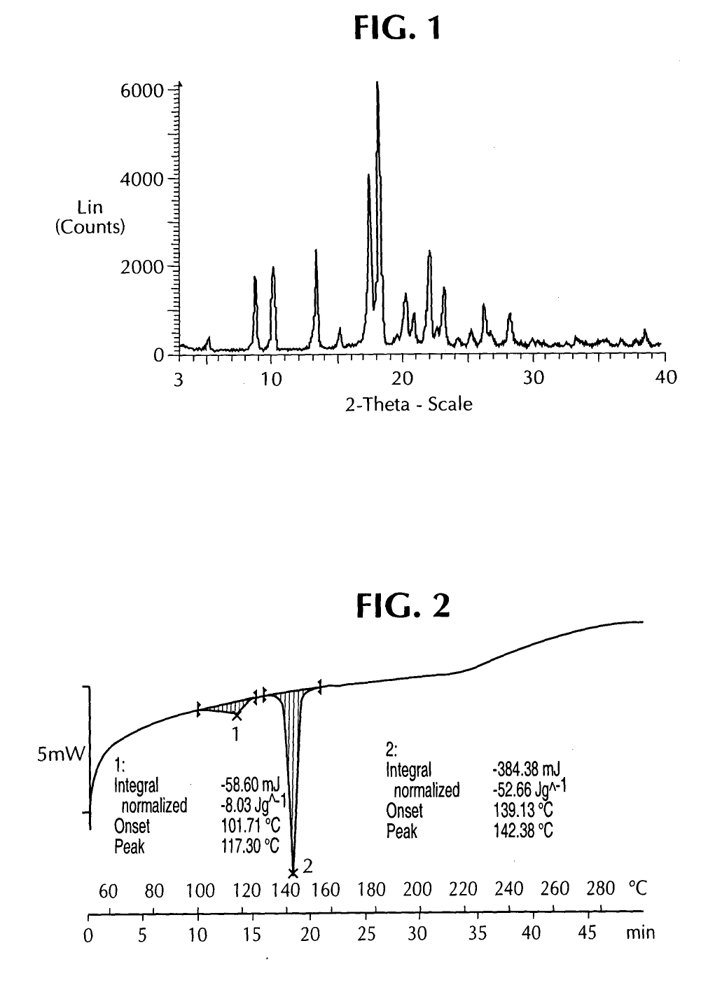

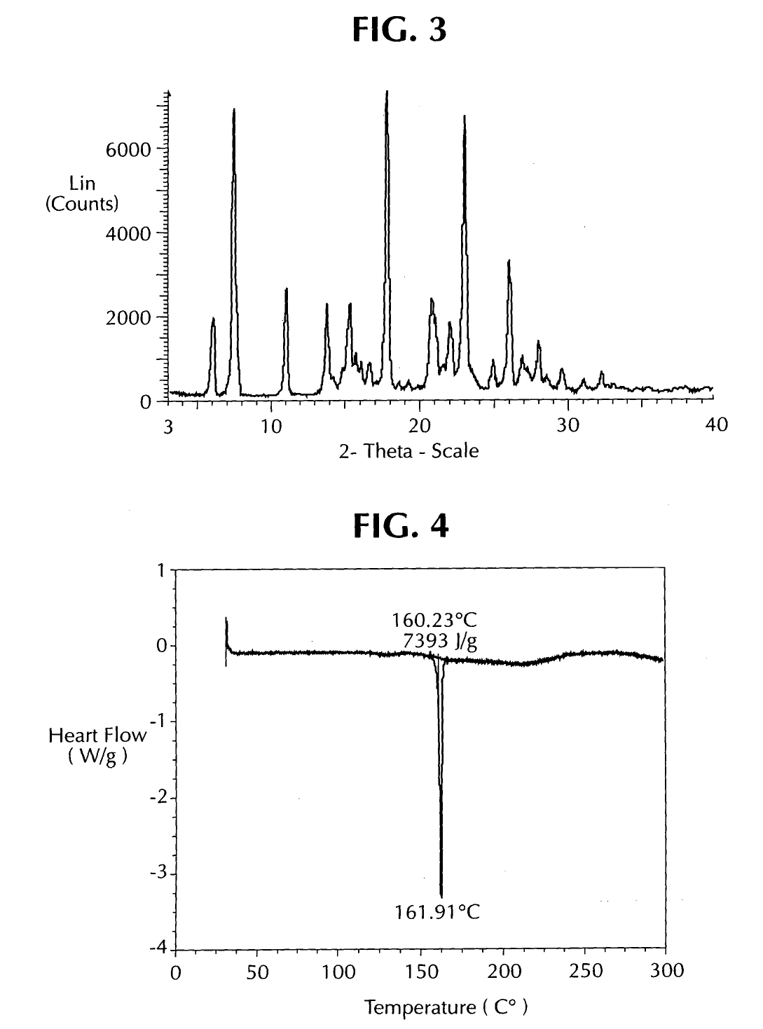

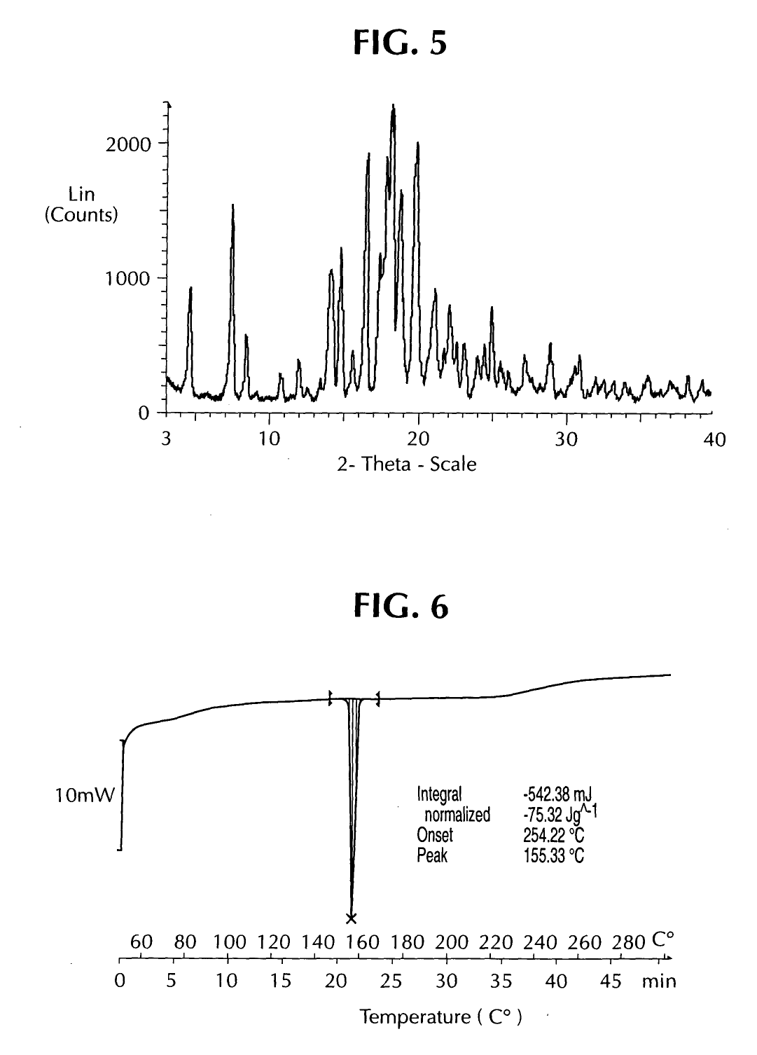

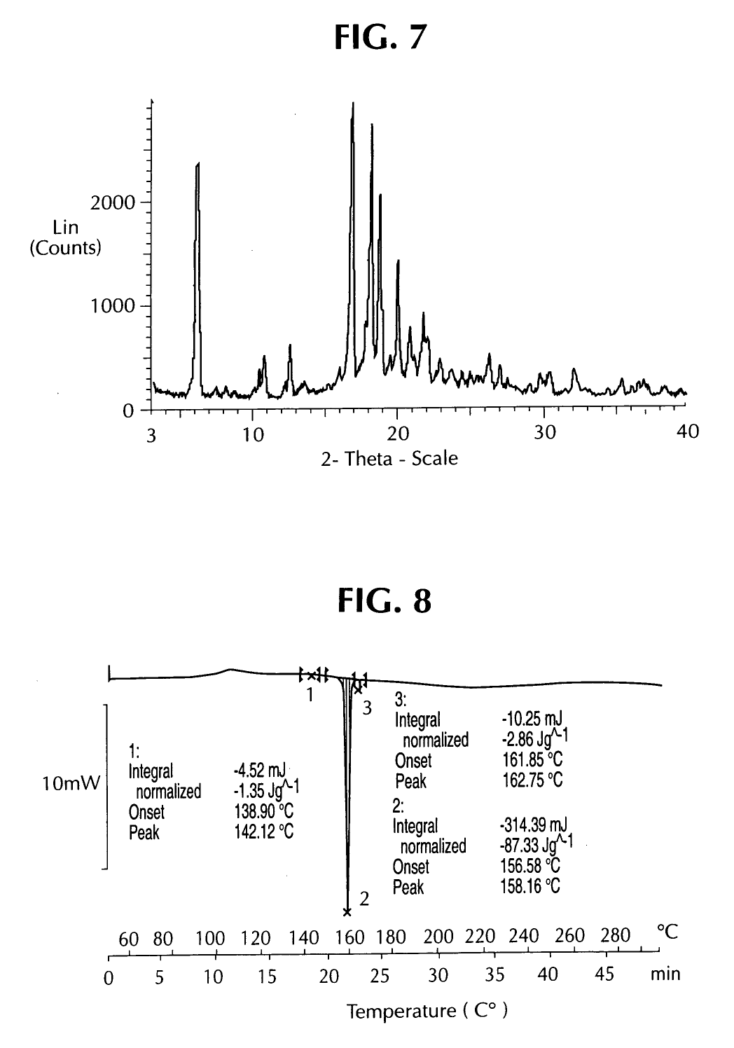

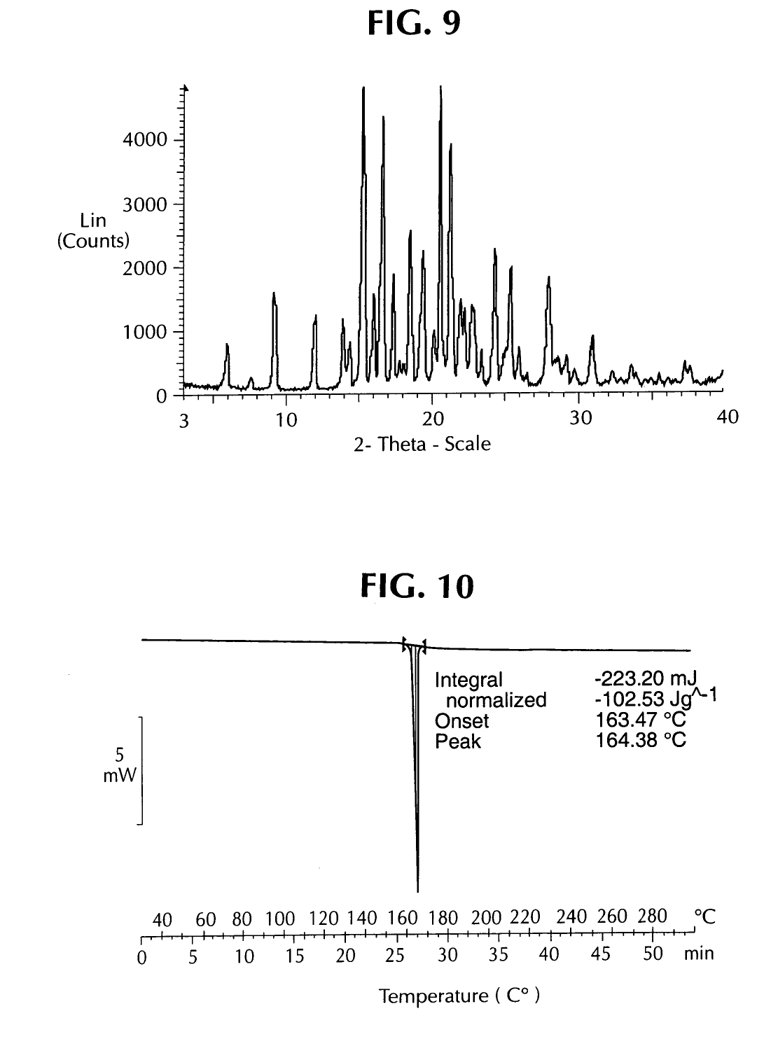

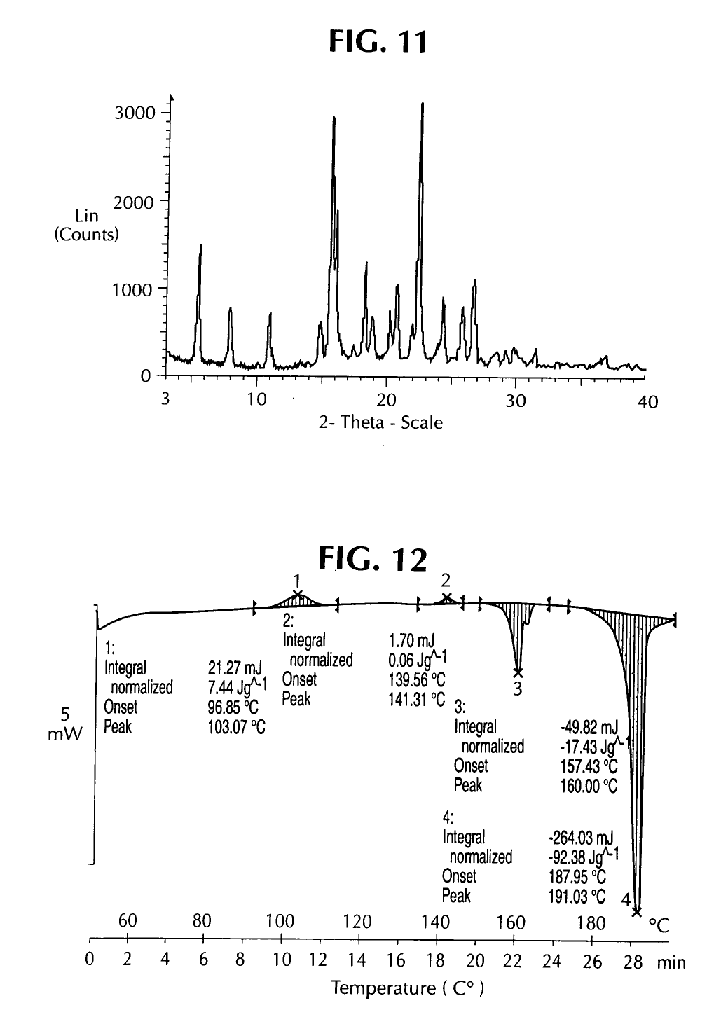

The results of a single crystal X-ray analysis are limited to, as the name implies, one crystal placed in the X-ray beam. Crystallographic data on a collection of powder crystals provides powder X-ray diffraction. Forms A-F have distinctive powder X-ray diffraction patterns. The powder X-ray diffraction patterns of Forms A-F are depicted, respectively, in FIGS. 1, 3, 5, 7, 9, and 11. The experimental conditions under which the powder X-ray diffraction was conducted are as follows: Cu anode; wavelength 1: 1.54056; wavelength 2: 1.54439 (Relative Intensity: 0.500); range #1-coupled: 3.000 to 40.000; step size: 0.040; step time: 1.00; smoothing width: 0.300; and threshold: 1.0.

The powder X-ray diffraction patterns display high intensity peaks, which are useful in identifying a specific crystal form. However, the relative intensities are dependent upon several factors, including, but not limited to, crystal size and morphology. As such, the relative intensity values may very from sample to sample. The powder X-ray diffraction values are generally accurate to within ±0.2 2-theta degrees, due to slight variations of instrument and test conditions. The powder X-ray diffraction patterns or a collective of the diffraction peaks for each of the crystal forms provide a qualitative test for comparison against uncharacterized crystals. The diffraction peaks detected with greater than 5% relative intensity are provided in Tables 2-7.

TABLE 2
Form A Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 5.1 | 5.7 | 19.5 | 6.4 | 25.3 | 7.8 |
| 8.8 | 28.4 | 20.2 | 21.9 | 26.3 | 17 |
| 10.1 | 32.5 | 20.8 | 14.3 | 26.8 | 7.9 |
| 13.3 | 38.5 | 22.0 | 37.6 | 28.2 | 14 |
| 15.1 | 9 | 22.6 | 9 | 33.3 | 5.3 |
| 17.5 | 65.5 | 23.2 | 23.7 | 38.6 | 7.8 |
| 18.2 | 100 | 24.2 | 5.3 | | |

TABLE 3
Form B Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 6.0 | 26.4 | 16.6 | 11 | 25.0 | 12.4 |
| 7.4 | 94.5 | 17.8 | 100 | 26.1 | 44.5 |
| 11.0 | 36 | 18.6 | 4.9 | 27.0 | 13.4 |
| 13.8 | 31 | 19.3 | 5.1 | 27.3 | 9.4 |
| 14.2 | 6.7 | 20.9 | 32.2 | 28.1 | 18.2 |
| 14.8 | 9.8 | 21.1 | 26.2 | 28.7 | 6.6 |
| 15.3 | 31.1 | 21.6 | 10.6 | 29.7 | 9.1 |
| 15.7 | 14.8 | 22.1 | 24.6 | 31.2 | 5 |
| 16.1 | 12.1 | 23.1 | 91.8 | 32.4 | 8 |

TABLE 4
Form C Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 4.6 | 40.2 | 19.0 | 37.5 | 28.3 | 9.5 |
| 7.4 | 68.4 | 19.7 | 89 | 29.0 | 22.9 |
| 8.4 | 25.1 | 20.6 | 17.9 | 30.3 | 11.4 |
| 10.8 | 12 | 21.1 | 40.5 | 30.6 | 15.7 |
| 11.9 | 17.1 | 21.7 | 21.4 | 31.0 | 19 |
| 12.6 | 7.6 | 22.1 | 35 | 32.1 | 11.7 |
| 13.4 | 10.8 | 22.6 | 22.9 | 32.6 | 10.7 |
| 14.1 | 46.6 | 23.1 | 22.3 | 33.3 | 10.7 |
| 14.8 | 53.9 | 24.1 | 18.7 | 34.1 | 9.8 |
| 15.6 | 20.4 | 24.5 | 22.1 | 34.4 | 8.1 |
| 16.4 | 84.7 | 25.0 | 34.7 | 35.4 | 9 |
| 17.4 | 52.5 | 25.6 | 16.4 | 35.7 | 11.9 |
| 17.8 | 84.1 | 26.2 | 13.6 | 37.2 | 10.7 |
| 18.1 | 100 | 27.3 | 18.9 | 38.4 | 12.5 |
| 18.7 | 73.2 | 27.7 | 11.4 | 39.3 | 11 |

TABLE 5
Form D Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 6.0 | 80.6 | 16.8 | 100 | 24.4 | 11.3 |
| 7.3 | 6.9 | 17.4 | 13.7 | 25.0 | 10.7 |
| 8.1 | 7.1 | 17.8 | 28.1 | 25.4 | 10.1 |
| 8.6 | 6 | 18.2 | 92.8 | 25.7 | 9.7 |
| 10.0 | 6.9 | 18.8 | 70 | 26.3 | 17.4 |
| 10.3 | 12.5 | 19.4 | 17.2 | 27.0 | 12.8 |
| 10.7 | 16.9 | 20.0 | 48.5 | 27.5 | 8.8 |
| 12.1 | 8.1 | 20.8 | 26.8 | 29.7 | 10.4 |
| 12.5 | 20.8 | 21.1 | 16.2 | 30.3 | 10.4 |
| 13.2 | 7.8 | 21.8 | 30.5 | 32.1 | 12.5 |
| 13.5 | 8.7 | 22.0 | 22.3 | 35.4 | 8.6 |
| 15.1 | 7.5 | 22.9 | 16 | 36.9 | 8.3 |
| 15.9 | 13 | 23.7 | 12.2 | | |

TABLE 6
Form E Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 5.9 | 16.5 | 19.4 | 46.8 | 28.0 | 37.6 |
| 7.6 | 5.4 | 20.1 | 20.5 | 28.7 | 11.3 |
| 9.2 | 33.2 | 20.6 | 99.5 | 29.2 | 12 |
| 12.0 | 25.7 | 21.2 | 82.2 | 29.8 | 6.9 |
| 13.9 | 24.2 | 21.9 | 30.7 | 30.9 | 18.3 |
| 14.3 | 17 | 22.3 | 27.4 | 32.3 | 6.3 |
| 15.2 | 100 | 22.8 | 27.9 | 33.6 | 8.4 |
| 16.0 | 32.2 | 23.4 | 14.4 | 33.9 | 5.8 |
| 16.6 | 90.1 | 24.3 | 46.9 | 35.6 | 5.5 |
| 17.3 | 38.6 | 24.9 | 12.3 | 37.3 | 10.1 |
| 17.7 | 10.3 | 25.4 | 40.4 | 37.6 | 8 |
| 18.0 | 9.4 | 26.0 | 14.4 | | |
| 18.5 | 52.8 | 26.5 | 5.8 | | |

TABLE 7
Form F Powder X-ray Diffraction Peaks

| Angle | I | Angle | I | Angle | I |
|---|---|---|---|---|---|
| 2-theta | (rel. %) | 2-theta | (rel. %) | 2-theta | (rel. %) |
| 5.4 | 47.5 | 17.4 | 10.2 | 24.2 | 29.2 |
| 7.8 | 24.9 | 18.1 | 41.9 | 25.4 | 10.4 |
| 10.8 | 22.4 | 18.7 | 21.5 | 25.8 | 25 |
| 14.7 | 19.6 | 20.1 | 23.4 | 26.6 | 35.6 |
| 15.6 | 94.3 | 20.6 | 32.5 | 29.8 | 11.2 |
| 15.9 | 61.2 | 21.8 | 19.1 | 31.4 | 10.8 |
| 16.6 | 9.7 | 22.3 | 100 | | |

Moreover, each form has high intensity peaks at two-theta:

Form A: 10.1, 13.3, 17.5, 18.2, and 22.0

Form B: 7.4, 11.0, 17.8, 23.1, and 26.1

Form C: 16.4, 17.8, 18.1, 18.7, and 19.7

Form D: 6.0, 16.8, 18.2, 18.8, and 20.0

Form E: 15.2, 16.6, 18.5, 20.6, and 21.2

Form F: 5.4, 15.6, 15.9, 18.1, and 22.3

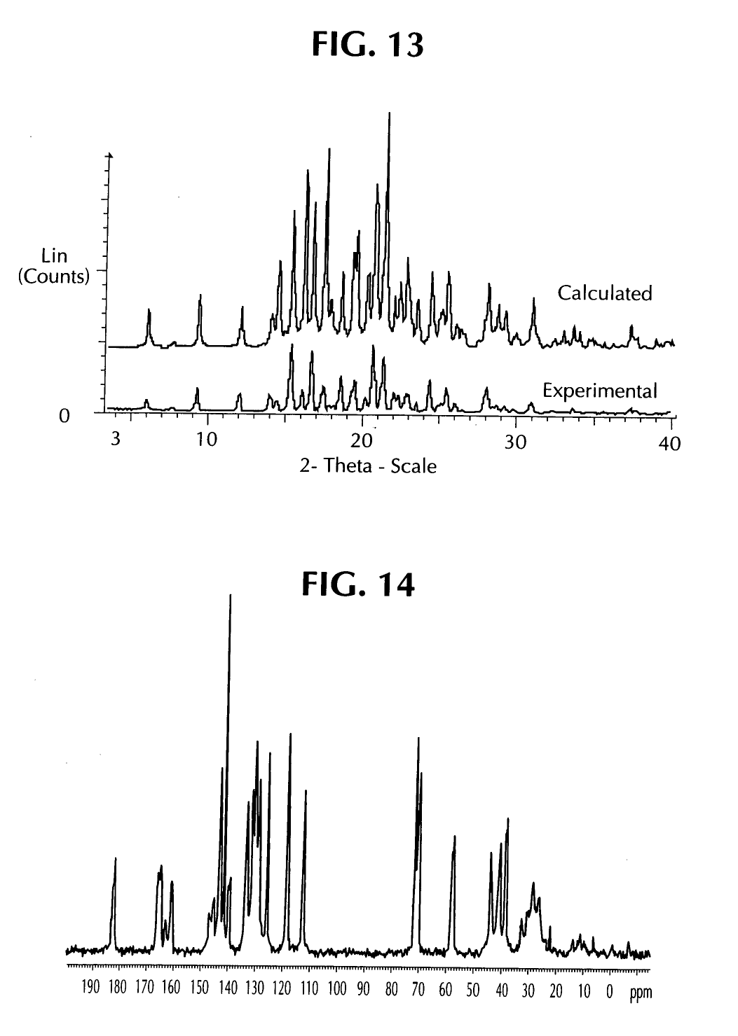

Single crystal structural data provide the cell dimensions and space group of a crystal form. These parameters are used as the basis to simulate an ideal powder pattern of that crystal form. The calculation can be done using SHELXTL Plus computer program, Reference Manual by Siemens Analytical X-ray Instrument, Chapter 10, p.179-181, 1990. Comparing the calculated powder X-ray diffraction pattern and the experimental representative powder x-ray diffraction pattern confirms whether a powder sample corresponds to an assigned single crystal structure. This procedure has been performed on the crystal form E and a match between the calculated and experimental representative powder x-ray diffraction patterns indicates the agreement between powder sample and the corresponding single crystal structure. (See FIG. 13 and Tables 1, 6 and 8). Table 8 provides the calculated diffraction peaks of form E based on the single crystal data.

TABLE 8
Form E powder X-ray Diffraction Peaks from Single Crystal Data*

| Angle | I* | Angle | I* | Angle | I* |
|---|---|---|---|---|---|
| 2-theta | (rel. %) | 2-theta | (rel. %) | 2-theta | (rel. %) |
| 6.0 | 15.6 | 20.1 | 31.9 | 28.5 | 9.8 |
| 7.6 | 2.7 | 20.6 | 68.9 | 28.7 | 19.4 |
| 9.2 | 22.2 | 21.3 | 100 | 29.2 | 16.2 |
| 12.0 | 17.3 | 22.0 | 22.9 | 29.9 | 7.3 |
| 14.0 | 14.9 | 22.3 | 28.2 | 31.0 | 21.7 |
| 14.4 | 36.9 | 22.8 | 38.9 | 31.3 | 6.6 |
| 14.8 | 7.1 | 23.0 | 25.6 | 31.9 | 2.9 |
| 15.3 | 58.6 | 23.5 | 21.5 | 32.3 | 5.4 |
| 16.0 | 75.5 | 24.4 | 32.6 | 32.9 | 8.2 |
| 16.6 | 62 | 25.1 | 16.8 | 33.6 | 9.7 |
| 17.4 | 84.9 | 25.4 | 32.6 | 34.0 | 8.2 |
| 17.8 | 21.3 | 26.0 | 10.9 | 37.3 | 11.2 |
| 18.1 | 9 | 26.3 | 9 | 37.6 | 6 |
| 18.5 | 32.5 | 26.5 | 7.1 | 38.1 | 2.8 |
| 19.2 | 40.3 | 28.0 | 27.9 | 38.9 | 4.6 |
| 19.4 | 50.1 | | | | |

*The calculated powder X-ray diffraction pattern represents all peaks with intensity % greater than 5%.
Peaks in italic/underlined were absent in the experimental pattern of Table 6 due to low intensity or unresolved with the adjacent peak within experimental error of ±0.2 degree 2-theta.

Differential Scanning Calorimetry (DSC) analysis was carried out on either TA Instruments DSC2920 or a Mettler DSC 821, calibrated with indium. DSC samples were prepared by weighing 2-4 mg of material in an aluminum pan with a pinhole. The sample was heated under nitrogen, at a rate of 5° C. per minute from about 30° C. to about 300° C. The onset temperature of the melting endotherm was reported as the melting temperature. The differential scanning calorimetry (DSC) thermograms for Forms A-F are shown, respectively, in FIGS. 2, 4, 6, 8, 10, and 12. The onset temperature of the melting endotherm is dependent on the rate of heating, the purity of the sample, crystal size and sample size, among other factors. Typically, the DSC results are accurate to within about ±2° C., preferably to within ±1.5° C. The thermograms may be interpreted as follows.

Referring to FIG. 2, Form A exhibits one major endotherm with an onset temperature of about 139° C.

Referring to FIG. 4, Form B exhibits an endotherm with an onset temperature of about 160° C.

Referring to FIG. 6, Form C exhibits an endotherm with an onset temperature of about 154° C.

Referring to FIG. 8, Form D exhibits one major endotherm with an onset temperature of about 156° C.

Referring to FIG. 10, Form E exhibits an endotherm with an onset temperature of about 163° C.

Referring to FIG. 12, Form F exhibits a main endotherm with an onset temperature of about 188° C.

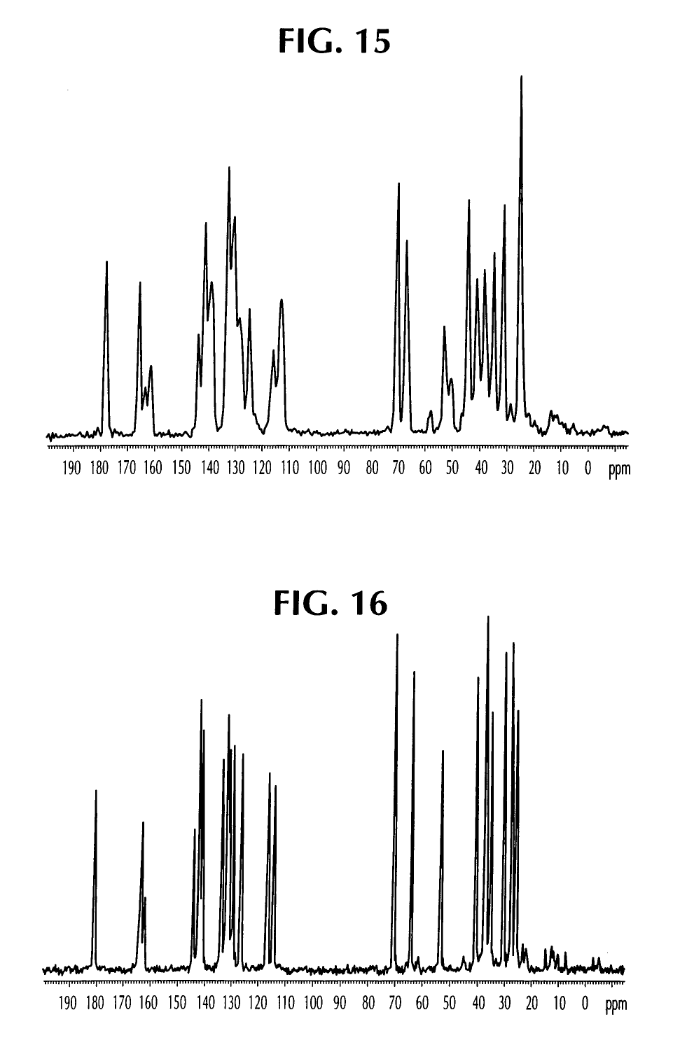

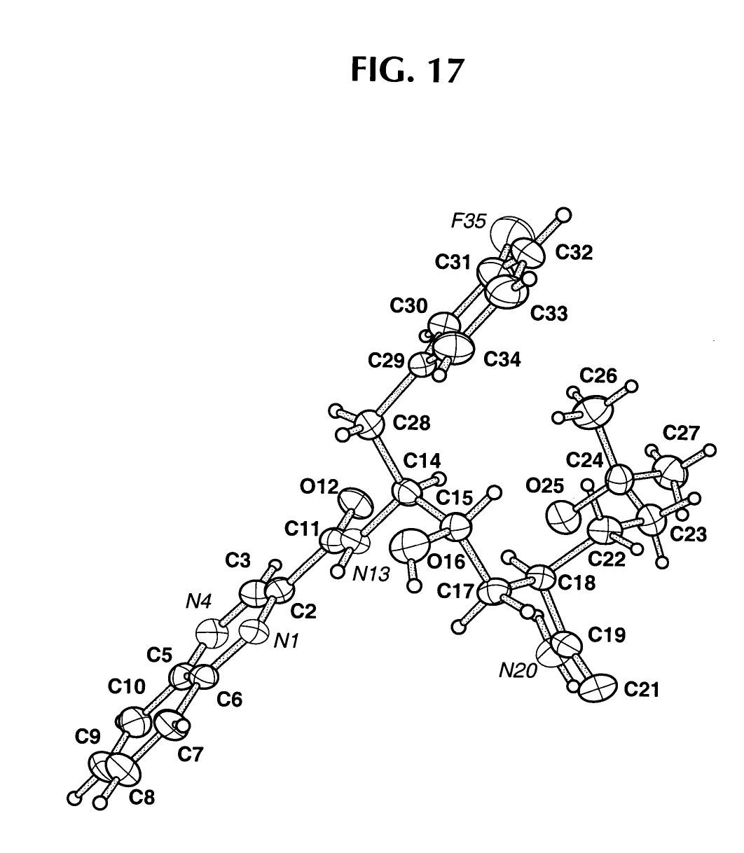

¹³C solid state nuclear magnetic resonance (ss-NMR) provides unique ¹³C chemical shifts spectra for each crystal form. Forms A, B and E have been analyzed with ss-NMR and are depicted, respectively, in FIGS. 14, 15, and 16. The experimental conditions under which the ss-NMR was conducted are as follows: collected on 11.75 T spectrometer (Bruker Biospin, Inc., Billerica, Mass.), corresponding to 125 MHz 13C frequency and acquired using cross-polarization magic angle spinning (CPMAS) probe operating at ambient temperature and pressure. 4 mm BL Bruker probes were employed, accommodating 75 mg of sample with maximum speed of 15 kHz. Data were processed with exponential line broadening function of 5.0 Hz. Proton decoupling of 100 kHz was used. Sufficient number of acquisitions were averaged out to obtain adequate signal-to-noise ratios for all peaks. Typically, 1500 scans were acquired with recycle delay of 4.5 s, corresponding to approximately 2-hour total acquisition time. Magic angle was adjusted using KBr powder according to standard NMR vendor practices. The spectra were referenced relative to the up-field resonance of adamantane (ADMNT) at 29.5 ppm. The spectral window minimally included the spectra region from 220 to −10 ppm. ¹³C chemical shifts between about 0 to 50 ppm and about 110 to 180 ppm may be useful in identifying the crystal form. The chemical shift data is dependent on the testing conditions (i.e. spinning speed and sample holder), reference material, and data processing parameters, among other factors. Typically, the ss-NMR results are accurate to within about ±0.2 ppm.

The ¹³C chemical shifts of Forms A, B, and E are shown in Table 9.

TABLE 9
¹³C ss NMR Chemical Shifts for Forms A, B and E

| A | B | E |
|---|---|---|
| 183.1* | 177.9 | 181.2 |
| 182.5 | 165.7 | 164.7 |
| 166.2 | 163.4 | 163.8 |
| 165.2 | 161.4 | 162.6 |
| 163.2 | 143.9 | 144.5 |
| 161.3 | 141.7 | 142.6 |
| 147.1 | 139.3 | 141.6 |
| 145.3 | 132.9 | 141.0 |
| 143.8* | 130.9 | 134.0 |
| 143.3 | 128.9 | 132.1 |
| 141.7 | 124.8 | 131.7 |
| 140.3 | 115.9 | 131.1 |
| 139.5 | 113.2 | 129.6 |
| 133.4 | 70.5 | 126.6 |
| 131.6 | 66.9 | 116.7 |
| 130.7 | 57.6** | 114.3 |
| 129.2 | 52.9 | 70.8 |
| 125.9 | 50.2 | 64.4 |
| 118.7 | 44.1 | 53.5 |
| 112.6 | 40.9 | 40.8 |
| 71.8 | 38.3 | 37.3 |
| 70.8 | 34.8 | 35.5 |
| 58.5 | 31.4 | 30.4 |
| 57.7 | 28.4** | 27.6 |
| 44.4 | 26.4 | 26.0 |
| 41.0 | | |
| 39.0 | | |
| 38.4 | | |
| 32.6 | | |
| 30.4 | | |
| 28.5 | | |
| 26.4 | | |

*Shoulders of the main peak
**Low intensity peaks

The crystalline Forms A-F may be prepared using any suitable method. Form A is a hemihydrate and as such, has approximately 1.5% water by weight. Forms B, C, D, E and F are all substantially anhydrous. Crystallization of the free base from a solvent system is carried out at a temperature from about 20° C. to about the solvent reflux temperature.

Form B may be formed by crystallizing quinoxaline-2-carboxylic acid [4carbamoyl-1-(3-fluorobenzyl)-2,7-dihydroxy-7-methyl-octyl]-amide free base in a solvent such as methylene chloride, methanol, or mixtures thereof. A solvent, such as methanol, is substantially removed in distillation and the product is crystallized therefrom. Preferably, the crystallization occurs from about room temperature to about 45° C. The crystallized product may be collected using any suitable method, including filtration and centrifugation. The collected crystallized product is then dried, preferably under vacuum at a temperature from about room temperature to about 45° C.

Form A may be formed by recrystallizing Forms B, C, D or F in isopropyl ether, toluene, tetrahydrofuran, isopropanol, ethanol, acetone, methanol, methyl ethyl ketone, water, or mixtures thereof at about room temperature to about 45° C. The presence of water in the crystallization medium facilitate conversion from anhydrous form B, C, D or F to form A.

Forms C and D may be formed by crystallizing quinoxaline-2-carboxylic acid [4-carbamoyl-1-(3-fluorobenzyl)-2,7-dihydroxy-7-methyl-octyl]-amide free base in acetonitrile at about room temperature and in mixtures of ethyl acetate, tetrahydrofuran and methyl tert-butyl ether above room temperature, preferably at about 45° C.

Forms E and F may prepared by recrystallization/reslurry of crystalline quinoxaline-2-carboxylic acid [4-carbamoyl-1-(3-fluorobenzyl)-2,7-dihydroxy-7methyl-octyl]-amide in ethyl acetate at about room temperature to about 45° C.

Quinoxaline-2-carboxylic acid [4-carbamoyl-1-(3-fluorobenzyl)-2,7-dihydroxy7-methyl-octyl]-amide of formula (Ia-3) is prepared as described in co-pending U.S. patent application Ser. No. 09/380,269, filed Feb. 5, 1998 and U.S. patent application Ser. No. 09/403,218, filed Jan. 18, 1999. Quinoxaline-2-carboxylic acid [4-carbamoyl-1-(3-fluorobenzyl)-2,7-dihydroxy-7-methyl-octyl]-amide of formula (Ia-3) may be further prepared according to Schemes 1 or 2.

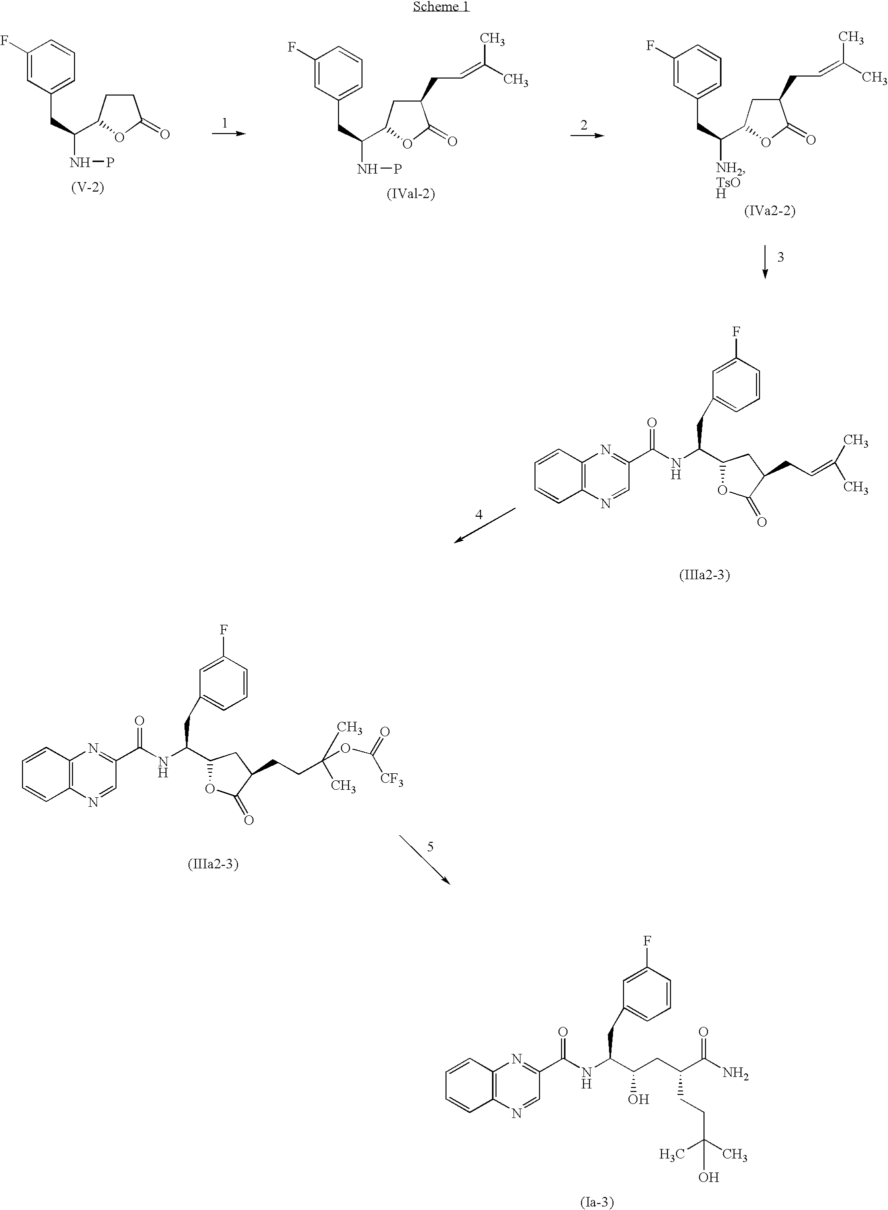

Quinoxaline-2-carboxylic acid [4-carbamoyl-1-(3-fluorobenzyl)-2,7-dihydroxy-7-methyl-octyl]-amide, (Ia-3) is formed by opening the lactone group and hydrolyzing the trifluoroacetate group of trifluoro-acetic acid 3-(5-{2-(3-fluoro-phenyl)-1-[(quinoxaline-2-carbonyl)-amino]-ethyl}-2-oxo-tetrahydro-furan-3-yl)-1,1-dimethylpropyl ester, (IIa2-3), as shown in step 5 of Scheme 1. This may be accomplished by reacting the compound IIa2-3 with ammonia either anhydrous in an organic solvent or as an aqueous solution of ammonium hydroxide added to a polar solvent at a temperature from about −10° C. to about 35° C., preferably at about 30° C. Suitable solvents include, alcohols, such as methanol, ethanol, or butanols; ethers such as tetrahydrofuran, glyme or dioxane; or a mixture thereof, including aqueous mixtures. Preferably the solvent is methanol. In one embodiment, the compound IIa2-3 is dissolved in methanol which has been saturated with ammonia gas. In another embodiment, the compound IIa2-3 in methanol is treated with ammonium hydroxide in tetrahydrofuran at room temperature.

The compound IIa2-3 is prepared in step 4 of Scheme 1 by hydrating the alkylene group of quinoxaline-2-carboxylic acid {2-(3-fluorophenyl)-1-[4-(3-methyl-but-2-enyl)-5-oxo-tetrahydrofuran-2-yl]-ethyl}-amide, (IIIa2-3). This hydration may occur by any suitable method. In one embodiment, the compound IIIa2-3 is reacted with trifluoroacetic acid in methylene chloride solution at room temperature to form the compound IIa2-3. The hydration may take several hours to complete at room temperature. A catalytic amount of sulfuric acid can be added to the reaction solution to increase the rate of reaction.

The compound IIIa2-3 is formed by coupling 5-[1-amino-2-(3-fluorophenyl)-ethyl]-3-(3-methyl-but-2-enyl)-dihydrofuran-2-one, tosylate salt, (IVa2-2) and quinoxaline-2-carboxylic acid or quinoxaline-2-carbonylchloride as shown in step 3 of Scheme 1. This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI)/dimethylaminopyridine (DMAP), and diethylphosphorylcyanide. The coupling is conducted in an inert solvent, preferably an aprotic solvent, such as acetonitrile, dichloromethane, chloroform, or N,N-dimethylformamide. One preferred solvent is methylene chloride. In one embodiment, quinoxaline acid is combined with methylene chloride, oxalyl chloride and a catalytic amount of N,N-dimethylformamide to form an acid chloride complex. The compound IVa2-2 is added to the acid chloride complex followed by triethylamine at a temperature from about 0° C. to about 25° C. to form the compound IIIa2-3.

The compound IVa2-2 is formed in step 2 of Scheme 1 by deprotecting the {2-(3-fluorophenyl)-1-[4-(3-methyl-but-2-enyl)-5-oxo-tetrahydrofuran-2-yl]-ethyl}-t-butoxycarbonyl-protected amine, (IVa1-2). Any suitable acidic deprotection reaction may be performed. In one example, an excess of p-toluenesulfonic acid hydrate in ethyl acetate is introduced to the compound IVa1-2 at room temperature. Suitable solvents include ethyl acetate, alcohols, tetrahydrofuran, and mixtures thereof. The reaction may proceed at ambient or elevated temperatures. Typically, the reaction is substantially complete within two and twelve hours. The resulting compound IVa2-2 may be crystallized and separated from the reaction mixture, and may be further purified to remove impurities by recrystallization from hot ethyl acetate.

The compound IVa1-2 is prepared by reacting 4-halo-2-methyl-2-butene; wherein halo may be iodo, bromo or chloro; with [2-(3-fluorophenyl)-1-(5-oxo-tetrahydrofuran-2-yl)-ethyl]-protected amine, (V-2), in the presence of a suitable base, as shown in Step 1 of Scheme 1. Exemplary bases include lithium dialkyl amides such as lithium N-isopropyl-N-cyclohexylamide, lithium bis(trimethylsilyl)amide, lithium di-isopropylamide, and potassium hydride. Suitable solvents include aprotic polar solvents such as ethers (such as tetrahydrofuran, glyme or dioxane), benzene, or toluene, preferably tetrahydrofuran. The aforesaid reaction is conducted at a temperature from about −78° C. to about 0° C., preferably at about −78° C. In one embodiment, alkylation of the lactone (V-2) is accomplished by reacting the lactone (V-2) with lithium bis(trimethylsilyl)amide and dimethylallyl bromide in tetrahydrofuran at a temperature from about −78° C. to about −50° C. Reaction times range from several hours or if an additive such as dimethyl imidazolidinone is present, the reaction may be complete in minutes.

Scheme 2 depicts an alternative reaction sequence for producing quinoxaline-2-carboxylic acid [4-carbamoyl-1-(3-fluorobenzyl)-2,7-dihydroxy-7-methyl-octyl]-amide (Ia-3).

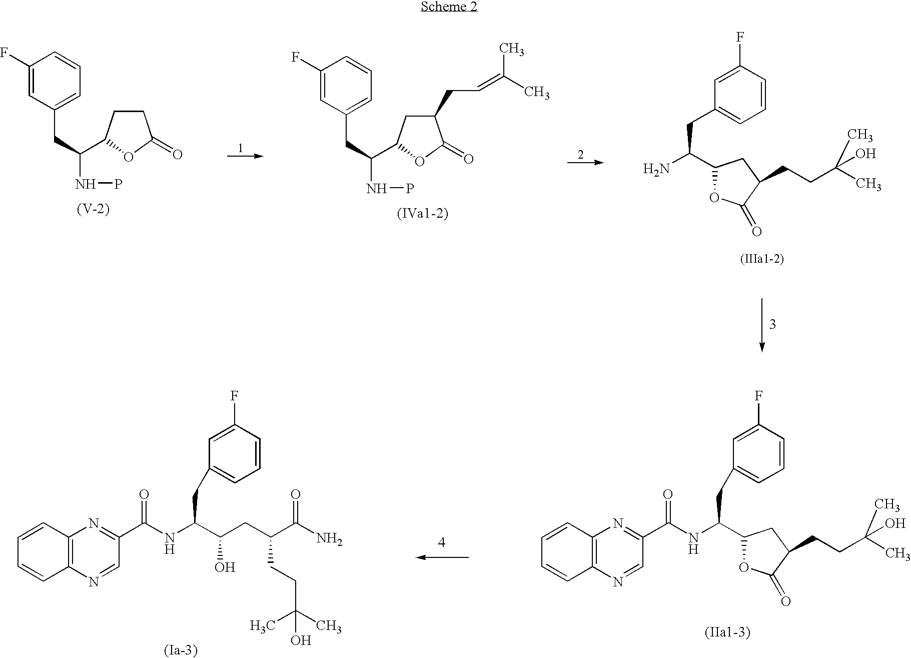

In Scheme 2, quinoxaline-2-carboxylic acid [4-carbamoyl-1-(3-fluorobenzyl)-2,7-dihydroxy-7-methyl-octyl]-amide, (Ia-3) is formed by opening the lactone group of the quinoxaline-2-carboxylic acid {2-(3-fluorophenyl)-1-[4-(3-hydroxy-3-methyl-butyl)-5-oxo-tetrahydro-furan-2-yl]-ethyl}-amide, (IIa1-3). This may be accomplished by reacting the compound IIa1-3 with ammonia either anhydrous in an organic solvent or as an aqueous solution of ammonium hydroxide add to a polar solvent at a temperature from about −10° C. to about 35° C., preferably at about 30° C. Suitable solvents include, alcohols, such as methanol, ethanol, or butanols; ethers such as tetrahydrofuran, glyme or dioxane, water; and mixture of such solvents. Preferably the solvent is methanol. In one embodiment, the compound IIa1-3 is dissolved in methanol which has been saturated with ammonia gas. In another embodiment, the compound IIa1-3 in methanol is treated with ammonium hydroxide in tetrahydrofuran at room temperature.

The compound IIa1-3 is prepared in step 3 of Scheme 2 by coupling 5-[1-amino-2-(3-fluoro-phenyl)-ethyl]-3-(3-hydroxy-3-methyl-butyl)-dihydro-furan-2-one, (IIIa1-2), and quinoxaline-2-carboxylic acid quinoxaline-2-carbonyl chloride. This coupling reaction is generally conducted at a temperature from about −30° C. to about 80° C., preferably from about 0° C. to about 25° C. The coupling reaction may occur with a coupling reagent that activates the acid functionality. Exemplary coupling reagents include dicyclohexylcarbodiimide/hydroxybenzotriazole (DCC/HBT), N-3-dimethylaminopropyl-N′-ethylcarbodiimide (EDC/HBT), 2-ethyoxy-1-ethoxycarbonyl-1,2-dihydroquinoline (EEDQ), carbonyl diimidazole (CDI), and diethylphosphorylcyanide. The coupling is conducted in an inert solvent, preferably an aprotic solvent, such as tetrahydrofuran, acetonitrile, dichloromethane, chloroform, or N,N-dimethylformamide. One preferred solvent is tetrahydrofuran. In one embodiment, quinoxaline acid is combined with CDI in anhydrous tetrahydrofuran and heated to provide the acyl imidazole. Compound IIIa1-2 is added to the acyl imidazole at room temperature to form the compound IIa1-3.

The compound IIIa1-2 is formed by hydrating the alkylene double bond and deprotecting the {2-(3-fluorophenyl)-1-[4-(3-methyl-but-2-enyl)-5-oxo-tetrahydrofuran-2-yl]-ethyl}-t-butoxycarbonyl-protected amine, (IVa1-2). Typically, this step is performed by reacting phosphoric acid with the compound IVa1-2. Preferably, this reaction occurs in any suitable solvent, such as non-alcoholic solvents. Two preferred solvents include tetrahydrofuran and dichloromethane. The reaction may take place at any suitable temperature, preferably from about −25° C. to about 120° C., more preferably from about 15° C. to about 40° C. Reaction time is dependent on temperature and batch size, amount other factors, but typically reaction time is from about 2 hours to about 14 hours.

The compound IVa1-2 preparation depicted as step 1 in Scheme 2 is the same chemical reaction using compound V-2, as depicted in step 1 of Scheme 1.

Unless indicated otherwise, the pressure of each of the above reactions is not critical. Generally, the reactions will be conducted at a pressure of about one to about three atmospheres, preferably at ambient pressure (about one atmosphere).

The compound of the formula Ia-3 is a potent antagonist of the CCR1 receptors, and as such, is useful in the treatment or prevention of autoimmune diseases (such as rheumatoid arthritis, type I diabetes (recent onset), inflammatory bowel disease, optic neuritis, psoriasis, multiple sclerosis, polymyalgia rheumatica, uveitis, and vasculitis), acute and chronic inflammatory conditions (such as osteoarthritis, adult respiratory distress syndrome, Respiratory Distress Syndrome of infancy, ischemia reperfusion injury, and glomerulonephritis), allergic conditions (such as asthma and atopic dermatitis), infection associated with inflammation (such as viral inflammation (including influenza and hepatitis) and Guillian-Barre), transplantation tissue rejection, atherosclerosis, restenosis, HIV infectivity (co-receptor usage), and granulomatous diseases (including sarcoidosis, leprosy and tuberculosis).

The activity of this compound of the invention can be assessed according to procedures known to those of ordinary skill in the art. Examples of recognized methods for determining CCR1 induced migration can be found in Coligan, J. E., Kruisbeek, A. M., Margulies, D. H., Shevach, E. M., Strober, W. editors: Current Protocols In Immunology, 6.12.1-6.12.3. (John Wiley and Sons, NY, 1991). One specific example of how to determine the activity of a compound for inhibiting migration is described in detail below.

Chemotaxis Assay:

The ability of compounds to inhibit the chemotaxis to various chemokines can be evaluated using standard 48 or 96 well Boyden Chambers with a 5 micron polycarbonate filter. All reagents and cells can be prepared in standard RPMI (BioWhitikker Inc.) tissue culture medium supplemented with 1 mg/ml of bovine serum albumin. Briefly, MIP-1α (Peprotech, Inc., P.O. Box 275, Rocky Hill N.J.) or other test agonists, were placed into the lower chambers of the Boyden chamber. A polycarbonate filter was then applied and the upper chamber fastened. The amount of agonist chosen is that determined to give the maximal amount of chemotaxis in this system (e.g., 1 nM for MIP-1α should be adequate).

THP-1 cells (ATCC TIB-202), primary human monocytes, or primary lymphocytes, isolated by standard techniques can then be added to the upper chambers in triplicate together with various concentrations of the test compound. Compound dilutions can be prepared using standard serological techniques and are mixed with cells prior to adding to the chamber.

After a suitable incubation period at 37 degrees centigrade (e.g. 3.5 hours for THP-1 cells, 90 minutes for primary monocytes), the chamber is removed, the cells in the upper chamber aspirated, the upper part of the filter wiped and the number of cells migrating can be determined according to the following method.

For THP-1 cells, the chamber (a 96 well variety manufactured by Neuroprobe) can be centrifuged to push cells off the lower chamber and the number of cells can be quantitated against a standard curve by a color change of the dye fluorocein diacetate.

For primary human monocytes, or lymphocytes, the filter can be stained with Dif Quik® dye (American Scientific Products) and the number of cells migrating can be determined microscopically.

The number of cells migrating in the presence of the compound are divided by the number of cells migrating in control wells (without the compound). The quotant is the % inhibition for the compound which can then be plotted using standard graphics techniques against the concentration of compound used. The 50% inhibition point is then determined using a line fit analysis for all concentrations tested. The line fit for all data points must have an coefficient of correlation (R squared) of greater than 90% to be considered a valid assay.

The compound of formula Ia-3 had an IC₅₀ of less than 25 μM, in the Chemotaxis assay. The compositions of the present invention may be formulated in a conventional manner using one or more pharmaceutically acceptable carriers. Thus, the active compounds of the invention may be formulated for oral, buccal, intranasal, parenteral (e.g., intravenous, intramuscular or subcutaneous) or rectal administration or in a form suitable for administration by inhalation or insufflation. The active compounds of the invention may also be formulated for sustained delivery.

For oral administration, the pharmaceutical compositions may take the form of, for example, tablets or capsules prepared by conventional means with pharmaceutically acceptable excipients such as binding agents (e.g., pregelatinized maize starch, polyvinylpyrrolidone or hydroxypropyl methylcellulose); fillers (e.g., lactose, microcrystalline cellulose or calcium phosphate); lubricants (e.g., magnesium stearate, talc or silica); disintegrants (e.g., potato starch or sodium starch glycolate); or wetting agents (e.g., sodium lauryl sulphate). The tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, methyl cellulose or hydrogenated edible fats); emulsifying agents (e.g., lecithin or acacia); non-aqueous vehicles (e.g., almond oil, oily esters or ethyl alcohol); and preservatives (e.g., methyl or propyl p-hydroxybenzoates or sorbic acid).

For buccal administration, the composition may take the form of tablets or lozenges formulated in conventional manner.

The active compounds of the invention may be formulated for parenteral administration by injection, including using conventional catheterization techniques or infusion. Formulations for injection may be presented in unit dosage form, e.g., in ampules or in multi-dose containers, with an added preservative. The compositions may take such forms as suspensions, solutions or emulsions in oily or aqueous vehicles, and may contain formulating agents such as suspending, stabilizing and/or dispersing agents. Alternatively, the active ingredient may be in powder form for reconstitution with a suitable vehicle, e.g., sterile pyrogen-free water, before use.

The active compounds of the invention may also be formulated in rectal compositions such as suppositories or retention enemas, e.g., containing conventional suppository bases such as cocoa butter or other glycerides.

For intranasal administration or administration by inhalation, the active compounds of the invention are conveniently delivered in the form of a solution or suspension from a pump spray container that is squeezed or pumped by the patient or as an aerosol spray presentation from a pressurized container or a nebulizer, with the use of a suitable propellant, e.g., dichlorodifluoromethane, trichlorofluoromethane, dichlorotetrafluoroethane, carbon dioxide or other suitable gas. In the case of a pressurized aerosol, the dosage unit may be determined by providing a valve to deliver a metered amount. The pressurized container or nebulizer may contain a solution or suspension of the active compound. Capsules and cartridges (made, for example, from gelatin) for use in an inhaler or insufflator may be formulated containing a powder mix of a compound of the invention and a suitable powder base such as lactose or starch.

A proposed dose of the active compounds of the invention for oral, parenteral or buccal administration to the average adult human for the treatment of the conditions referred to above (e.g., rheumatoid arthritis) is 0.1 to 1000 mg of the active ingredient per unit dose which could be administered, for example, 1 to 4 times per day.

Aerosol formulations for treatment of the conditions referred to above (e.g., rheumatoid arthritis) in the average adult human are preferably arranged so that each metered dose or “puff” of aerosol contains 20 μg to 1000 μg of the compound of the invention. The overall daily dose with an aerosol will be within the range 0.1 mg to 1000 mg. Administration may be several times daily, for example 2, 3, 4 or 8 times, giving for example, 1, 2 or 3 doses each time.

The active agents can be formulated for sustained delivery according to methods well known to those of ordinary skill in the art. Examples of such formulations can be found in U.S. Pat. Nos. 3,538,214, 4,060,598, 4,173,626, 3,119,742, and 3,492,397.

The compounds of the invention can also be utilized in combination therapy with other therapeutic agents such as with immunosuppressant agents such as cyclosporin A and FK-506, Cellcept®, rapamycin, leuflonamide or with classical anti-inflammatory agents (e.g. cyclooxygenase/lipoxegenase inhibitors) such as tenidap, aspirin, acetaminophen, naproxen and piroxicam, steroids including prednisone, azathioprine and biological agents such as OKT-3, anti IL-2 monoclonal antibodies (such as TAC).

Experimental

The following examples are put forth so as to provide those of ordinary skill in the art with a complete disclosure and description of how the compounds, compositions, and methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.) but some errors and deviations should be accounted for. Unless indicated otherwise, percent is percent by weight given the component and the total weight of the composition, temperature is in ° C. or is at ambient temperature, and pressure is at or near atmospheric. Commercial reagents were utilized without further purification. Other abbreviations used herein are defined as follows: g is grams, L is liter, mg is milligram, and mL is milliliter.

Note that all numbers provided herein are approximate, but effort have been made to ensure accuracy with respect to numbers (e.g., amounts, temperature, etc.); however some errors and deviations should be accounted for.

### EXAMPLE 1

### Preparation of quinoxaline-2-carboxylic acid [4(R)-carbamoyl-1(S)-(3-fluoro-benzyl)-2(S),7-dihydroxy-7-methyl-octyl]-amide (Ia-3), Form B

2.78 kg of quinoxaline-2-carboxylic acid [4(R)-carbamoyl-1(S)-(3-fluoro-benzyl)-2(S),7-dihydroxy-7-methyl-octyl]-amide free base was dissolved in 10 volumes of methylene chloride and 1 volume of methanol to produce a slurry. One volume of methanol was added to create a solution, and the solution was filtered to produce a substantially speck free solution. This solution was then distilled azeotropically under atmospheric pressure until the over-head temperature reached about 40° C. The contents were granulated and filtered to produce approximately 2.5 kg, resulting in a 90% yield of form B.

### EXAMPLE 2

### Preparation of quinoxaline-2-carboxylic acid [4(R)-carbamoyl-1(S)-(3-fluoro-benzyl)-2(S),7-dihydroxy-7-methyl-octyl]-amide (Ia-3), Form E

A portion of the wet filter cake from example 1 was charged to a vessel and 10 volumes of ethyl acetate were added to the vessel. The mixture was heated to reflux, and 5 volumes of ethyl acetate were then distilled off under atmospheric pressure. Five volumes of hexanes were then added, and the resulting mixture was granulated. Upon confirmation of polymorph form E, the mixture was filtered and rinsed with 1:1 mixture of ethyl acetate/hexanes. The filter cake was blown dry with nitrogen, collected and dried under vacuum at 40-45° C.

### EXAMPLE 3

### Preparation of quinoxaline-2-carboxylic acid [4(R)-carbamoyl-1(S)-(3-fluoro-benzyl)-2(S),7-dihydroxy-7-methyl-octyl]-amide (Ia3). Form A

A portion of the wet filter cake from example 1 was charged to a vessel and 10 volumes of ethyl acetate and 1 volume of methanol were added to the vessel to dissolve the compound. The solution was heated to reflux, and ethyl acetate was added thereby displacing the methanol. Water was added and the resulting mixture was granulated and filtered. The filter cake was blown dry with nitrogen, collected and dried under vacuum at about 30° C. for about 24 hours. The crystalline product of form A was achieved with about 93% yield.

### EXAMPLE 4

### Preparation of quinoxaline-2-carboxylic acid [4(R)-carbamoyl-1(S)-(3-fluoro-benzyl)-2(S), 7-dihydroxy-7-methyl-octyl]-amide (Ia3), Form A

114 mg of a combination of quinoxaline-2-carboxylic acid [4(R)-carbamoyl-1(S)-(3-fluoro-benzyl)-2(S),7-dihydroxy-7-methyl-octyl]-amide forms B, C, and D were added to 3-5 mL hexanes with a trace amount of water and stirred at room temperature of six days. The contents were filtered and dried on pump to produce approximately 96 mg of form A.

### EXAMPLE 5

### Preparation of quinoxaline-2-carboxylic acid [4(R)-carbamoyl-1(S)-(3fluoro-benzyl)-2(S),7-dihydroxy-7-methyl-octyl]-amide (Ia3), Form B

1.7 g of quinoxaline-2-carboxylic acid [4(R)-carbamoyl-1(S)-(3-fluoro-benzyl)-2(S),7-dihydroxy-7-methyl-octyl]-amide free base (oil form) was heated in ethyl acetate and cooled to give an amorphous solid. This sold was heated to reflux in ethyl acetate and hexanes were added until turbid. The mixture was cooled and a crystalline solid formed. Filtered the crystalline solid to give 1.0 g of form B.

Throughout this application, various publications are referenced. The disclosures of these publications in their entireties are hereby incorporated by reference into this application for all purposes.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. A crystal form of quinoxaline-2-carboxylic acid [4-carbamoyl-1-(3-fluorobenzyl)-2,7-dihydroxy-7-methyl-octyl]-amide comprising form A, form B, form C, form D, form E or form F.

2. A crystal form of quinoxaline-2-carboxylic acid [4-carbamoyl-1-(3-fluorobenzyl)-2,7-dihydroxy-7-methyl-octyl]-amide form A having a powder X-ray diffraction pattern comprising peaks expressed in degrees two-theta at approximately 5.1, 8.8, 10.1, 13.3, 15.1, 17.5, 18.2, 19.5, 20.2, 20.8, 22.0, 22.6, 23.2, 24.2, 25.3, 26.3, 26.8, 28.2, 33.3, and 38.6.

3. A crystal form of quinoxaline-2-carboxylic acid [4-carbamoyl-1-(3-fluorobenzyl)-2,7-dihydroxy-7-methyl-octyl]-amide form A having a solid state nuclear magnetic resonance spectrum comprising ¹³C chemical shifts expressed in parts per million at approximately 182.5, 166.2, 165.2, 163.2, 39.0, 38.4, 32.6, 30.4, 28.5, and 26.4.

4. A crystal form of quinoxaline-2-carboxylic acid [4-carbamoyl-1-(3-fluorobenzyl)-2,7-dihydroxy-7-methyl-octyl]-amide form B having a powder X-ray diffraction pattern comprising peaks expressed in degrees two-theta at approximately 6.0, 7.4, 11.0, 13.8, 14.2, 14.8, 15.3, 15.7, 16.1, 16.6, 17.8, 18.6, 19.3, 20.9, 21.1, 21.6, 22.1, 23.1, 25.0, 26.1, 27.0, 27.3, 28.1, 28.7, 29.7, 31.2, and 32.4.

5. A crystal form of quinoxaline-2-carboxylic acid [4-carbamoyl-1-(3-fluorobenzyl)-2,7-dihydroxy-7-methyl-octyl]-amide form B having a solid state nuclear magnetic resonance spectrum comprising ¹³C chemical shifts expressed in parts per million at approximately 177.9, 165.7, 163.4, 161.4, 40.9, 38.3, 34.8, 31.4, and 26.4.

6. A crystal form of quinoxaline-2-carboxylic acid [4-carbamoyl-1-(3-fluorobenzyl)-2,7-dihydroxy-7-methyl-octyl]-amide form C having a powder X-ray diffraction pattern comprising peaks expressed in degrees two-theta at approximately 4.6, 7.4, 8.4, 10.8, 11.9, 12.6, 13.4, 14.1, 14.8, 15.6, 16.4, 17.4, 17.8, 18.1, 18.7, 19.0, 19.7, 20.6, 21.1, 21.7, 22.1, 22.6, 23.1, 24.1, 24.5, 25.0, 25.6, 26.2, 27.3, 27.7,28.3, 29.0, 30.3, 30.6, 31.0, 32.1, 32.6, 33.3, 34.1, 34.4, 35.4, 35.7, 37.2, 38.4, and 39.3.

7. A crystal form of quinoxaline-2-carboxylic acid [4-carbamoyl-1-(3-fluorobenzyl)-2,7-dihydroxy-7-methyl-octyl]-amide form D having a powder X-ray diffraction pattern comprising peaks expressed in degrees two-theta at 6.0, 7.3, 8.1, 8.6, 10.0, 10.3, 10.7, 12.1, 12.5, 13.2, 13.5, 15.1, 15.9, 16.8, 17.4, 17.8, 18.2, 18.8, 19.4, 20.0, 20.8, 21.1, 21.8, 22.0, 22.9, 23.7 24.4, 25.0, 25.4, 25.7, 26.3, 27.0, 27.5, 29.7, 30.3, 32.1, 35.4, and 36.9.

8. A crystal form of quinoxaline-2-carboxylic acid [4-carbamoyl-1-(3-fluorobenzyl)-2,7-dihydroxy-7-methyl-octyl]-amide form E having a powder X-ray diffraction pattern comprising peaks expressed in degrees two-theta at approximately 5.9, 7.6, 9.2, 12.0, 13.9, 14.3, 15.2, 16.0, 16.6, 17.3, 17.7, 18.0, 18.5, 19.4, 20.1, 20.6, 21.2, 21.9, 22.3, 22.8, 23.4, 24.3, 24.9, 25.4, 26.0, 26.5, 28.0, 28.7, 29.2, 29.8, 30.9, 32.3, 33.6, 33.9, 35.6, 37.3, and 37.6.

9. A crystal form of quinoxaline-2-carboxylic acid [4-carbamoyl-1-(3-fluorobenzyl)-2,7-dihydroxy-7-methyl-octyl]-amide form E having a solid state nuclear magnetic resonance spectrum comprising ¹³C chemical shifts expressed in parts per million at approximately 181.2, 164.7, 163.8, 162.6, 40.8, 37.3, 35.5, 30.4, 27.6, and 26.0.

10. The crystal form of quinoxaline-2-carboxylic acid [4-carbamoyl-1-(3-fluorobenzyl)-2,7-dihydroxy-7-methyl-octyl]-amide according to claim 1 further having a differential scanning calorimetry thermogram comprising an endothermic event with an onset temperature of about 163° C. using a heating rate of about 5° C. per minute from about 30° C. to about 300° C.

11. A crystal form of quinoxaline-2-carboxylic acid [4-carbamoyl-1-(3-fluorobenzyl)-2,7-dihydroxy-7-methyl-octyl]-amide form F having a powder X-ray diffraction pattern comprising peaks expressed in degrees two-theta at approximately 5.4, 7.8, 10.8, 14.7, 15.6, 15.9, 16.6, 17.4, 18.1, 18.7, 20.1, 20.6, 21.8, 22.3, 24.2, 25.4, 25.8, 26.6, 29.8, and 31.4.

12. A crystal form of quinoxaline-2-carboxylic acid [4-carbamoyl-1-(3-fluorobenzyl)-2,7-dihydroxy-7-methyl-octyl]-amide form E, wherein the crystal has an empirical formula of C₂₆H₃₁N₄O₄F; a formula weight of about 482.55; a melt temperature of about 298(2) K; wavelength of about 1.54178 Å; orthorhombic crystal system; a space group P2(1)2(1)2(1); unit cell dimensions of a about 6.7678(2) Å α=90°, b about 12.6136(3) Å β=90°, and c about 29.4200(7) Å γ=90°; volume of about 2511.48(11) Å³ and Z of 4.

13. A pharmaceutical composition for treating or preventing a disorder or condition that can be treated or prevented by antagonizing the CCR1 receptor in a subject, comprising an amount of a compound of any of claims 1-12 effective in such disorders or conditions, or a pharmaceutically acceptable salt thereof, and a pharmaceutically acceptable carrier.

14. A method of preparing crystalline quinoxaline-2-carboxylic acid [4-carbamoyl1-(3-fluorobenzyl)-2,7-dihydroxy-7-methyl-octyl]-amide comprising:
a) mixing quinoxaline-2-carboxylic acid [4-carbamoyl-1-(3-fluorobenzyl)-2,7-dihydroxy-7-methyl-octyl]-amide free base in a solvent mixture of methanol and methylene chloride to create mixture 1;
b) distilling mixture 1 to substantially remove methanol to form mixture 2;
c) crystallizing mixture 2 in a solvent system comprising ethyl acetate.

15. The method according to claim 14, wherein the solvent system of step (c) further comprises methanol.
